(19) United States
(12) Patent Application Publication
(10) Pub. No.: US 2024/0228451 A1
(43) Pub. Date: Jul. 11, 2024

(54) METHODS OF SYNTHESIZING LIPSTATIN DERIVATIVES

(71) Applicant: Panafina, Inc., Morrisville, NC (US)

(72) Inventors: Zeeshan Kamal, San Ramon, CA (US); Apparao Bokka, Santa Clara, CA (US); Gopalakrishna Akurathi, Santa Rosa, CA (US); Gary Richard Lee, Belmont, CA (US)

(21) Appl. No.: 18/556,727

(22) PCT Filed: Apr. 21, 2022
(86) PCT No.: PCT/US2022/025704

§ 371 Date: Oct. 23, 2023

Related U.S. Application Data
(60) Provisional application No. 63/178,728, filed on Apr. 23, 2021.

(51) Int. Cl. C07D 305/12; C07C 51/347; C07C 51/487; C07C 67/14; C07C 67/31; C07C 67/313; C07D 309/30; C07D 309/32

(52) U.S. Cl. CPC C07D 305/12; C07C 51/347; C07C 51/487; C07C 67/14; C07C 67/31; C07C 67/313; C07D 309/30; C07D 309/32

(57) ABSTRACT

Provided according to embodiments of the invention are methods of forming a lipstatin derivative, (S)-1-((2S,3S)-3-ethyl-4-oxooxetan-2-yl) tridecan-2-yl formyl-L-alaninate. Such methods include one or more reaction steps that include the Mitsunobu coupling of N-formyl L-alanine in the presence of di-t-butyl azodicarboxylate (DBAD) and triphenylphosphine. Compounds formed by a method of the invention, compositions that include a compound formed by a method of the invention, and methods of inhibiting lipase activity and/or treating pancreatitis using a compound formed by a method of the invention are also provided.

### STATEMENT OF PRIORITY

This application claims the benefit of U.S. Provisional Application Ser. No. 63/178,728, filed Apr. 23, 2021, the entire contents of which are incorporated by reference herein.

### FIELD OF THE INVENTION

The present invention relates to methods of synthesizing pharmaceutical compounds. The present invention also relates to small molecule lipase inhibitors and methods of synthesizing the same. In particular, the present invention relates to methods of synthesizing lipstatin derivatives.

### Background of the Invention

Pancreatitis is an inflammation of the pancreas that may occur when digestive enzymes become activated while still in the pancreas, thus irritating cells of the pancreas and causing inflammation. The annual incidence of acute pancreatitis is approximately 20 to 40 per 100,000 people, and this incidence has increased over recent decades. See, Yadav et al., Epidemiology of Pancreatitis, GI Epidemiology: Diseases and Clinical Methodology, 2^{nd} Ed., 2014, Blackwell Publishing. Furthermore, severe acute pancreatitis has a mortality rate of approximately 29%. Munoz et al., Am Fam Physician (2000) 62: 164-74. As such, new methods of treating pancreatitis are being investigated.

Lipase inhibitors are a class of compounds that have been commercialized as anti-obesity agents. Lipstatin, a natural product isolated from Streptomyces toxytricini, was an early known lipase inhibitor. A saturated derivative of lipstatin, orlistat, was developed by Hoffman-La Roche and marketed as an anti-obesity drug using the brand names, Xenical® and Alli®. Lipase inhibitors may also be useful for treating other disorders, such as pancreatitis.

One lipstatin derivative is (S)-1-((2S,3S)-3-ethyl-4-oxooxetan-2-yl)tridecan-2-yl formyl-L-alaninate, also referred to herein as Compound I-A, shown below.

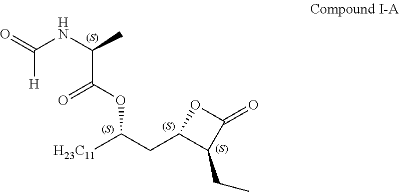

Current methods of synthesizing Compound I-A include the synthetic procedure shown in FIG. 1. While Compound I-A may be produced by such methods, the methods may not be suitable for large scale and/or cGMP manufacturing. In addition, such methods may have chemical processing issues, including strong sulfur/thiol odors that are difficult to contain on scale-up and significant health hazards due to the use of corrosive aqueous hydrofluoric acid. In addition, the overall synthesis may produce low to moderate yields and may require seven chromatography purification steps. A such, new methods for synthesizing Compound I-A may be desirable.

### SUMMARY OF THE INVENTION

Provided according to embodiments of the invention are methods for synthesizing a compound of Formula I:

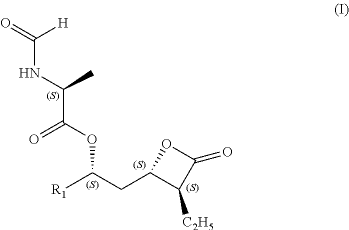

wherein R₁ is a C₅-C₁₅ alkyl (e.g., C₁₁ alkyl). In some embodiments, the methods include contacting a compound of INT 12 with N-formyl L-alanine, in the presence of di-t-butyl azodicarboxylate (DBAD) and triphenylphosphine, to form the compound of Formula I.

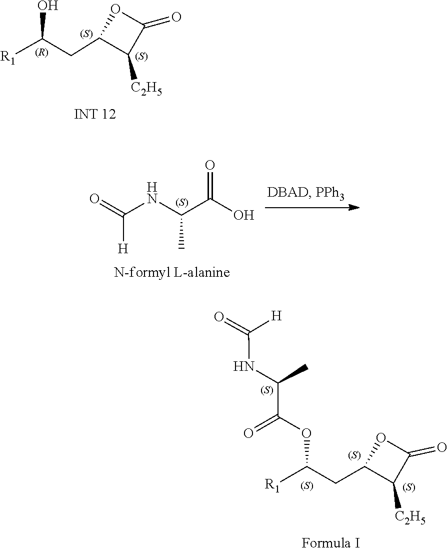

In some embodiments of the invention, provided are methods for synthesizing a compound of INT 2. In some embodiments, such methods include contacting a compound of INT1 with a ruthenium (R)-BINAP catalyst and hydrogen gas in a reaction mixture in a pressure vessel to form a compound of INT 2:

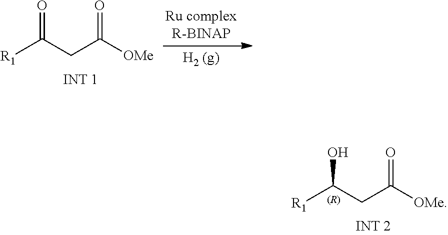

In some embodiments, the pressure of the hydrogen gas in the pressure vessel is less than or equal to 200 psi (e.g., in a range of 100 to 200 psi). Further, in some embodiments, a volume ratio of dead space to the reaction mixture in the pressure vessel is 3:1 or more.

Also provided according to embodiments of the invention are methods for synthesizing the compound of Formula I:

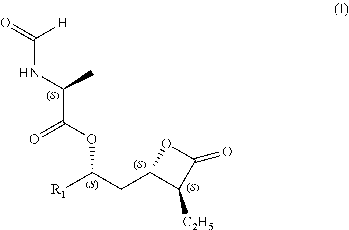

wherein R₁ is a C₅-C₁₅ alkyl (e.g., C₁₁ alkyl), that include:
(a) contacting a compound of 1-A with a compound of 1-B, optionally in the presence of magnesium, to form a compound of INT 1:

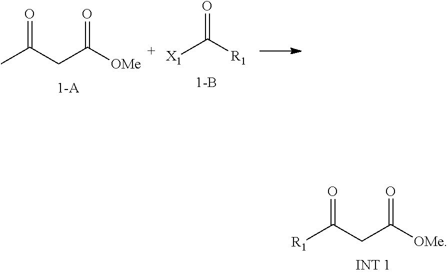

wherein X₁ is a halo (e.g., Cl or Br);
(b) contacting the compound of INT1 with a ruthenium (R)-BINAP catalyst and hydrogen gas to form the compound of INT 2:

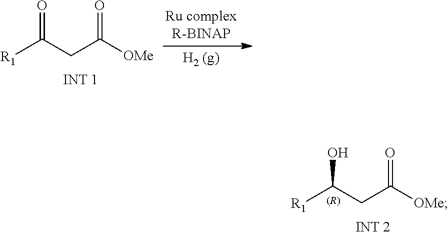

(c) contacting the compound of INT 2 with a compound of 3-A to form a compound of INT 3:

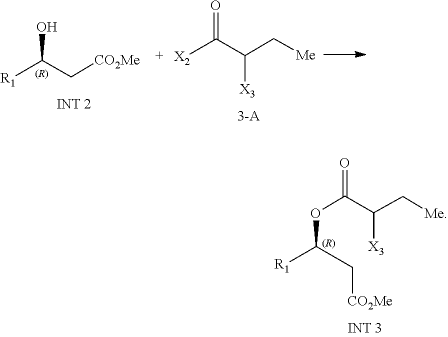

wherein X₂ and X₃ are each independently halo (e.g., Br);
(d) contacting the compound of INT 3 with a Grignard reagent to form a compound of INT 4:

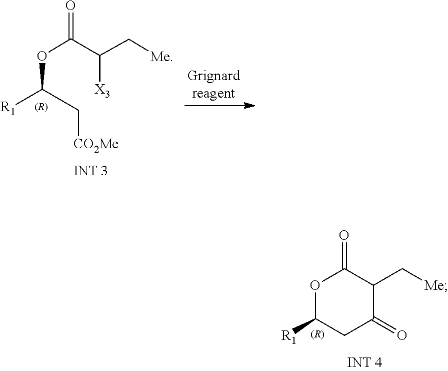

(e) contacting the compound of INT 4 with hydrogen gas in the presence of a Raney Ni catalyst to form a compound of INT 5:

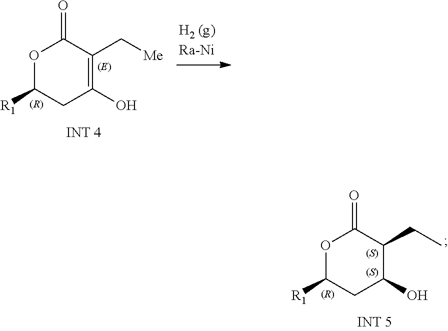

(f) contacting the compound of INT 5 with a first protecting agent to form a compound of INT 6:

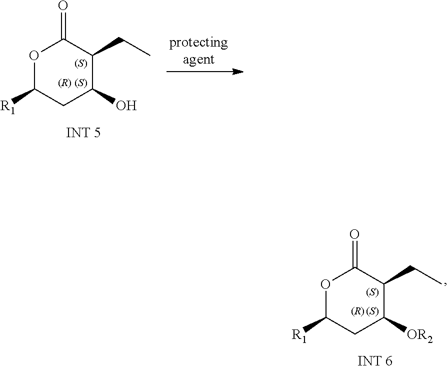

wherein R₂ is a protecting group (e.g., THP);
(g) contacting the compound of INT 6 with a hydroxide (e.g., from a hydroxide salt such as NaOH) to form a compound of INT 7:

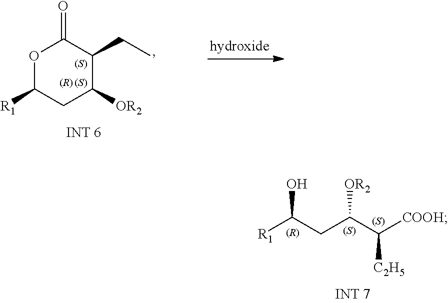

(h) protecting a free hydroxy group on a compound of INT 7 by reacting INT 7 with a second protecting agent to form a compound of INT 8, and then deprotecting a protected hydroxy group on the compound of INT 8 by contacting INT 8 with acid to form the compound of INT 9:

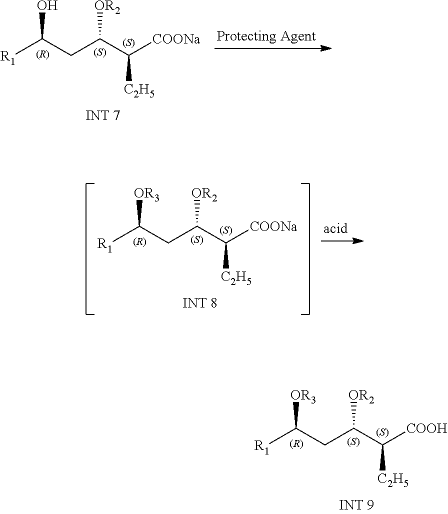

wherein R₃ is a protecting group (e.g., benzyl);
(i) optionally, purifying the compound of INT 9 by contacting the compound of INT 9 with (S)-(−)-1-phenyethylamine to form a compound of INT 10:

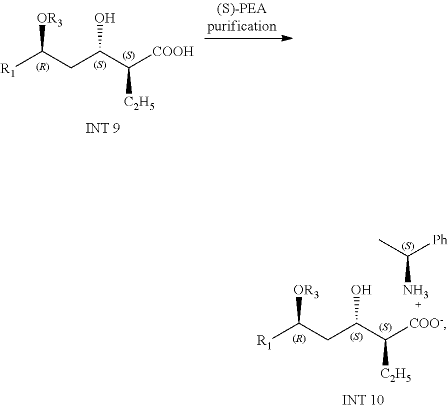

crystallizing and isolating INT 10A, and then contacting INT 10 with an acid (e.g., HCl) to form INT 9 (purified):

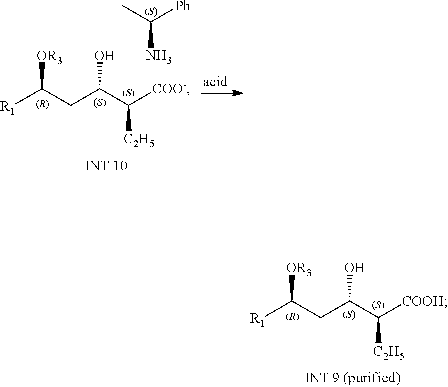

(j) dehydrating the compound of INT 9 or the compound of INT 9 (purified) with a dehydrating agent to form a compound of INT 11:

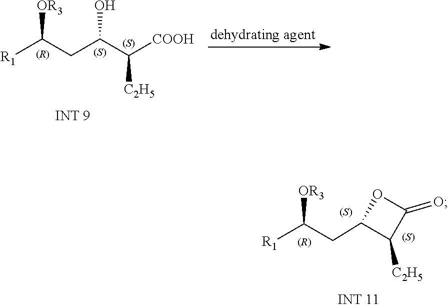

(k) deprotecting the compound of INT 11 to form a compound of INT 12:

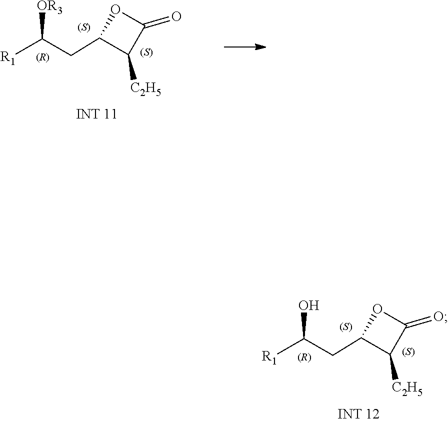

and
(l) contacting the compound of INT 12 with N-formyl L-alanine, in the presence of di-t-butyl

azodicarboxylate (DBAD) and triphenylphosphine, to form the compound of Formula I:

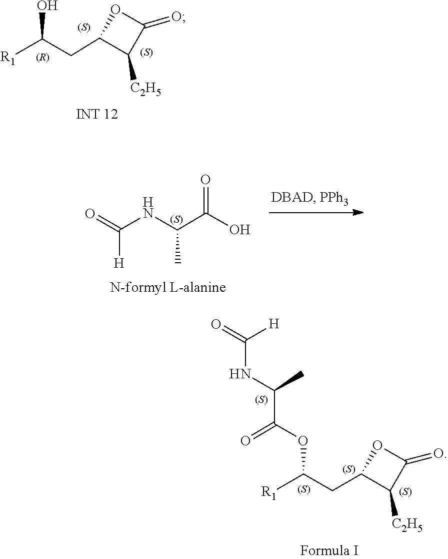

Further provided according to embodiments of the invention are compounds of Formula I formed by a method of the invention or a pharmaceutically acceptable salt thereof. Also provided are compositions that include a compound of the invention and a pharmaceutically acceptable carrier.

Additionally, provided according to embodiments of the invention are methods of inhibiting lipase activity in a subject in need thereof that include administering a therapeutically effective amount of a compound of the invention or a pharmaceutically acceptable salt thereof and/or a composition of the invention to the subject, thereby inhibiting lipase activity in the subject.

Also provided according to embodiments of the invention are methods of treating pancreatitis in a subject in need thereof that include administering a therapeutically effective amount of a compound of the invention or a pharmaceutically acceptable salt thereof and/or a composition of of the invention to the subject, thereby treating pancreatitis in the subject.

These and other aspects of the invention are set forth in more detail in the description of the invention below.

### BRIEF DESCRIPTION OF THE DRAWINGS

FIG. 1 is a synthetic scheme for a prior art method of producing Compound I-A.

FIG. 2 is an ¹H NMR spectrum of INT 1.

FIG. 3 is an ¹H NMR spectrum of enantiomerically enriched INT 2.

FIG. 4 is an ¹H NMR spectrum of racemic INT 2.

FIG. 5 shows chiral HPLC data for enantiomerically enriched INT 2.

FIGS. 6A and 6B are photographs of thin layer chromatography (TLC) plates showing the elution of starting material (SM) and product for INT 3 under UV lamp (FIG. 6A) and with PMA stain (FIG. 6B) when 20% ethyl acetate/heptane was used as the eluent.

FIG. 7 is an ¹H NMR spectrum of a crude reaction mixture of INT 3.

FIGS. 8A and 8B are photographs of thin layer chromatography (TLC) plates showing the elution of starting material (SM) and product for INT 4 for crude mixture (IPC, FIG. 8A) and for the crystallized product (FIG. 8B).

FIG. 9 is an ¹H NMR spectrum of INT 4, which shows keto-enol tautomerism.

FIG. 10 shows an experimental set-up for Raney Ni hydrogenation.

FIGS. 11A and 11B are photographs of thin layer chromatography (TLC) plates showing the elution of starting material and product for INT 5 for crude (FIG. 11A) and crystalline product (FIG. 11B).

FIG. 12 is an ¹H NMR spectrum of crystalline INT 5.

FIG. 13 is an LC-MS spectrum of INT 6.

FIG. 14 is an LC-MS spectrum of INT 7.

FIG. 15 is an LC-MS spectrum of INT 8.

FIG. 16 is an LC-MS spectrum of INT 9.

FIG. 17 is an LC-MS spectrum of INT 10.

FIG. 18 is an LC-MS spectrum of the mother liquor that includes PEA-purified INT 8.

FIG. 19 is an LC-MS spectrum of INT 9 converted from PEA-purified INT 8 in the mother liquor.

FIG. 20 is an LC-MS spectrum of INT 10 converted from PEA-purified INT 8 in the mother liquor.

FIG. 21 is an LC-MS spectrum of INT 10 after recrystallization.

FIG. 22 is an LC-MS spectrum of INT 9 (purified).

FIG. 23 is an LC-MS spectrum of INT 11.

FIG. 24 is an LC-MS spectrum of INT 12.

FIG. 25 is an LC-MS spectrum of the crude mixture including Compound I-A (RABI-767).

FIG. 26 is an LC-MS spectrum of the crude mixture including Compound I-A after treatment with TFA/DCM.

FIG. 27 is an LC-MS spectrum of the crude mixture including Compound I-A after treatment with TFA/DCM when synthesized on a large scale.

FIG. 28A is a photograph of a column packing set up for purifying Compound I-A.

FIG. 28B is a photograph of TLC plates stained with PMA showing the elution of Compound I-A with various other impurities, using 50/50 heptane/ethyl acetate eluent.

FIG. 29 is an LC-MS spectrum of Compound I-A.

FIG. 30 is an ¹H NMR spectrum of Compound I-A.

FIG. 31 shows a HPLC readout and spectrum for Compound I-A.

FIG. 32 shows HPLC data for Compound I-A.

FIG. 33 shows a crystal structure for the Compound I-A.

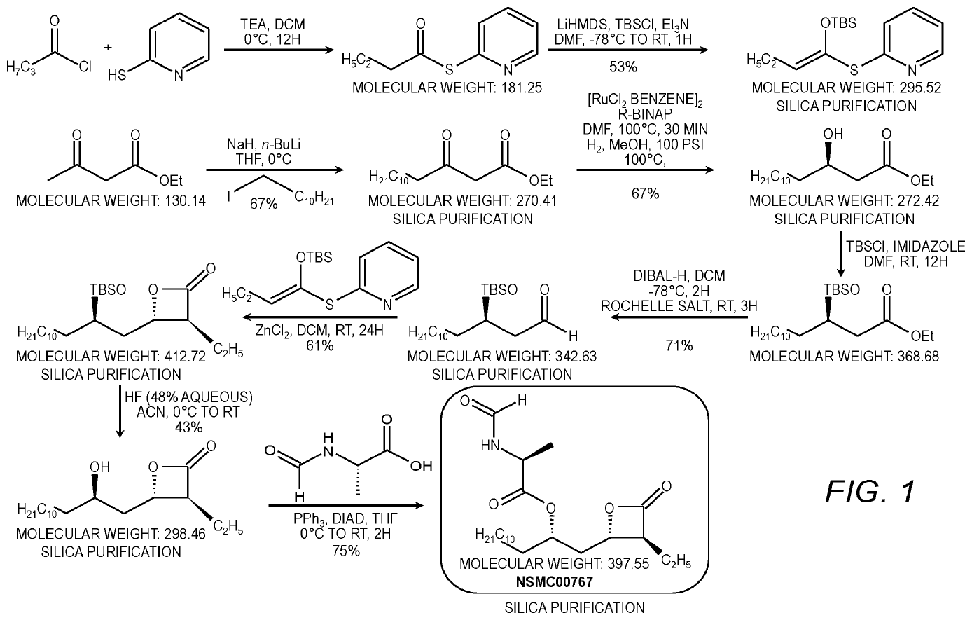

### DETAILED DESCRIPTION OF THE EXAMPLE EMBODIMENTS

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

“Halo” as used herein refers to any suitable halogen, including —F, —Cl, —Br, and —I.

“Hydroxy” or “hydroxyl,” as used herein, refer to an —OH group. A “free hydroxy group” is an —OH group that is not protected. A “protected hydroxy group” is a hydroxy group that is bound (e.g., covalently bound) to a protecting group.

“Alkyl” as used herein alone or as part of another group, refers to a straight or branched chain hydrocarbon containing from 1 to 15 carbon atoms. “Lower alkyl” as used herein alone or as part of another group, refers to a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, n-dodecyl, tridecyl, tetradecyl, pentadecyl and the like. As such, a C₅-C₁₅ alkyl includes straight and branched saturated alkyl including, for example, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, n-dodecyl, tridecyl, tetradecyl, and pentadecyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description unless otherwise indicated.

The compounds described herein can also or further contain linkages wherein bond rotation is restricted about that particular linkage, e.g., restriction resulting from the presence of a ring or double bond (e.g., carbon-carbon bonds, carbon-nitrogen bonds such as amide bonds). Accordingly, all cis/trans and E/Z isomers and rotational isomers are included in the present description. Unless otherwise indicated, the chemical designation of a compound encompasses the mixture of all possible stereochemically isomeric forms of that compound.

Preparation of compounds described herein can involve the protection and deprotection of various chemical groups. The chemistry of protecting groups can be found, for example, in T. W. Greene and P. G. M. Wuts, Protective Groups in Organic Synthesis, 3rd Ed., Wiley & Sons, Inc., New York (1999).

Reactions can be monitored, and products identified, by methods known in the art. For example, product formation can be monitored by spectroscopic means, such as nuclear magnetic resonance spectroscopy, infrared spectroscopy, spectrophotometry (e.g., UV-visible), mass spectrometry, and/or by chromatographic methods such as high performance liquid chromatography (HPLC), liquid chromatography-mass spectroscopy (LCMS), or thin layer chromatography (TLC). Compounds can be purified by a variety of methods known to those of skill in the art unless otherwise indicated.

“Treatment” as used herein means any manner in which one or more of the symptoms of a disease or disorder are ameliorated or otherwise beneficially altered. Treatment also encompasses any pharmaceutical use of the compositions herein, such as use for treating pancreatitis. As used herein, amelioration of the symptoms of a particular disorder by administration of a particular compound or pharmaceutical composition refers to any lessening, whether permanent or temporary, lasting or transient that can be attributed to or associated with administration of the composition.

Provided according to embodiments of the invention are methods for synthesizing a compound of Formula I:

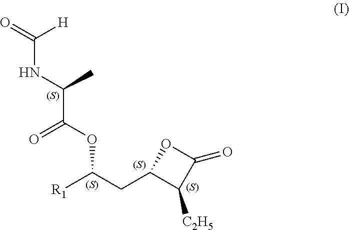

wherein R₁ is a C₅-C₁₅ alkyl (e.g., a C₅-C₁₅ straight chain alkyl). R₁ may thus be a C₅, C₆, C₇, C₈, C₉, C₁₀, C₁₁, C₁₂, C₁₃, C₁₄, or C₁₅ alkyl or any range defined therebetween. In particular embodiments, R₁ is a C₁₁ alkyl (e.g., C₁₁H₂₃) such as when the compound of Formula I is Compound I-A.

Provided below are method steps in the synthesis of a compound of Formula I. Methods of the invention may include one or more of these method steps. As such, in some embodiments, certain method steps may be omitted and any single step itself may be considered a method of the invention. For example, a method according to an embodiment of the invention may include steps 2-14, steps 3-14, steps 4-14, steps 5-14, steps 6-14, steps 7-14, steps 8-14, steps 9-14, steps 10-14, steps 11-14, steps 12-14, step 13-14, and step 14 alone. Additionally, any combination of intermediate steps may be a method of the invention, (e.g., steps 2-4, steps 4-6, steps 8-10). In some embodiments, each of the method steps described herein is present in a method of the invention, e.g., in a method to synthesize Compound I-A.

### Method Step 1

In some embodiments of the invention, a compound of Formula 1-A is contacted with a compound of Formula 1-B to form a compound of Formula INT 1, as shown below:

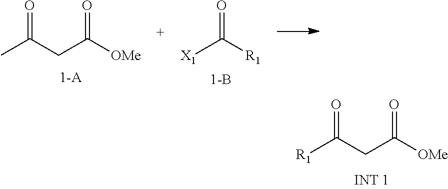

wherein X₁ is halo (e.g., Cl or Br) and R₁ is C₅-C₁₅ alkyl (e.g., C₅-C₁₅ straight chain alkyl). As such, R₁ may be a C₅, C₆, C₇, C₈, C₉, C₁₀, C₁₁, C₁₂, C₁₃, C₁₄, or C₁₅ alkyl or any range defined therebetween. In particular embodiments, R₁ is a C₁₁ alkyl (e.g., C₁₁H₂₃).

In some embodiments, the compound of 1-A contacts the compound of 1-B in the presence of an alkali metal such as magnesium, and the compound of INT 1 is formed by aliphatic acylation. In some embodiments, the alkali metal (e.g., magnesium) is first reacted with methanol to form an alkali metal methoxide solution (optionally in toluene) into which the compound of 1-A is added. The compound of of 1-B dissolved in a solvent (e.g., toluene) may then be added over time such as via an addition funnel. In some embodiments, the reaction is quenched with methanol and distilled. A strong acid such as a mineral acid, e.g., hydrochloric acid, sulfuric acid, and/or phosphoric acid, may then be added. The reaction may then be purified by any suitable method, such as, by washing with water, drying, and/or concentrating the compound of INT 1. Optionally, the compound of INT 1 may then be recrystallized (e.g., with methanol) to further purify the compound.

### Method Step 2

In some embodiments of the invention, a compound of INT 1 is contacted with a ruthenium R-BINAP catalyst and hydrogen gas in a stereoselective ketone reduction to form the compound of INT 2:

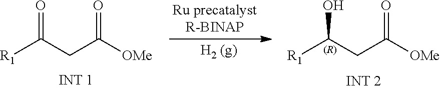

wherein R₁ is defined above.

In some embodiments, the R-BINAP, (R)-(2,2′-bis(diphenylphosphino)-1,1′-binaphthyl, and the ruthenium metal form a catalyst in situ when a ruthenium precatalyst and the R-BINAP ligands are combined in an inert atmosphere and then added to the reaction mixture. In some embodiments, the ruthenium precatalyst is a ruthenium chloride compound such as [RuCl₂ benzene]₂. In some embodiments, the ruthenium precatalyst and R-BINAP are combined in a degassed solvent and an inert atmosphere and heated (e.g., to 100° C.) to form the active catalyst solution. The compound of INT 1 is dissolved in a suitable solvent (in an inert environment) to form a reaction mixture and the active catalyst solution is added to the reaction mixture. In some embodiments, the compound of INT 1 and the active catalyst are added to an autoclave or pressure vessel that is pressured with hydrogen gas and heated to form a compound of INT 2. In some embodiments, the pressure of the hydrogen gas in the autoclave or pressure vessel is at a pressure of less than or equal to 200 psi (e.g., in a range of 100 psi to 200 psi). The deadspace in the pressure vessel may also affect the purity and enantioselectivity of the resulting compound of INT 2. As such, in some embodiments, the ratio of the deadspace to the reaction mixture in the pressure vessel is 3:1 or more (e.g., 4:1, 5:1; or 6:1). The crude compound of INT 2 may be further purified by extraction and/or washing, and in some embodiments, recrystallized by dissolution in an organic solvent and cooled.

In some embodiments, in order to assess the enantiopurity of the compound of INT 2, the compound may be derivatized to form a Mosher's ester. In some embodiments, the formation of the Mosher's ester is performed using Mosher's acid (α-methoxy-α-trifluormethylphenylacetic acid (MTPA)) using a catalyst (e.g., 4-dimethylaminopyridine (DMAP)) and a dehydrating agent (e.g., dicyclohexylcarbodiimide (DCC)). The Mosher's ester may then be analyzed by ¹H NMR to determine if the compound of INT 2 is enantiomerically enriched.

In some embodiments, in order to assess the enantiopurity of the compound of INT 2, the compound may be derivatized with benzoyl chloride, such as in the presence of DMAP and N,N-diisopropylethylamine (DIPEA). The derivative thus formed may be analyzed by chiral HPLC to determine the entantiomeric excess (% ee). In some embodiments, the compound of INT 2 has a % ee of at least 90% (90-99% ee), at least 95% (95-99% ee), or at least 97% (97-99% ee).

### Method Step 3

In some embodiments of the invention, a compound of INT2 is contacted with compound 3-A to form a compound of INT 3:

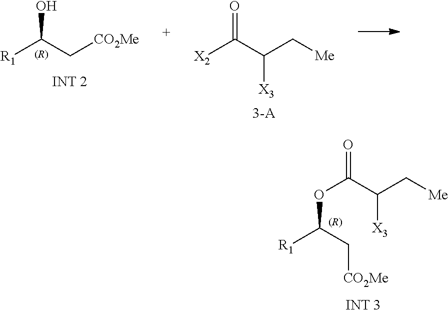

wherein R₁ is defined above and X₂ and X₃ are each independently halo (e.g., Br or Cl).

In some embodiments, the O-acylation reaction of the compound of INT 2 occurs in the presence of a catalyst (e.g., DMAP) and potassium bicarbonate (KHCO₃) under Schotten-Baumann reaction conditions (two phase reaction including water and an organic solvent such as toluene). Such a reaction may be performed at low temperatures such as in a range of 0° C. to 15° C. After the reaction is complete, the temperature may be increased, and water added to the reaction vessel to hydrolyze any unreacted compound 3-A. In some embodiments, the compound of INT 3 thus formed may be washed with water and/or the organic phase extracted and concentrated.

### Method Step 4

In some embodiments of the invention, a compound of INT 3 is contacted with a Grignard reagent (a Reformatsky reaction) to cyclize and form a compound of INT 4:

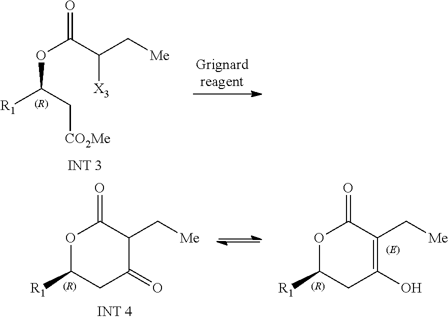

wherein R₁ and X₃ are defined above, and INT 4 exists as a tautomer.

In some embodiments, the Grignard reagent is a tertiary Grignard agent such as t-butylMgBr, t-amylMgBr, and the like. In some embodiments, a reaction mixture including the compound of INT 3 and the Grignard reagent are charged to a reactor with organic solvent (e.g., THF) slowly over time until the reaction forms a compound of INT 4. The reaction mixture may optionally be distilled to remove a portion of the organic solvent, the product cooled, and/or a cold solution of citric acid may be added to facilitate precipitation.

In some embodiments, the product may be washed and/or recrystallized to purify the compound of INT 4.

### Method Step 5

In some embodiments of the invention, a compound of INT4 is contacted with hydrogen gas, in the presence of a Raney Ni catalyst. to form a compound of INT 5:

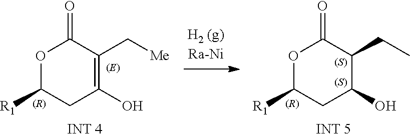

wherein R₁ is defined above.

In some embodiments, the hydrogen gas is present in a reaction vessel at a pressure in a range of 0.5 atm to 5 atm, and in some embodiments, at approximately 1 atm. In some embodiments, the Raney Ni catalyst is freshly prepared prior to the reaction (not commercially purchased), such as immediately prior to the reaction. In some embodiments, the Raney Ni (e.g., freshly prepared Raney Ni) is added to a reaction vessel under inert conditions and the compound of INT 4 is then added to the reaction vessel. Hydrogen gas may then be passed through the reaction mixture via a gas diffusion sparger while stirring. Once the reaction is complete (e.g., as determined by TLC or LCMS), the Raney Ni may be allowed to settle, and the supernatant removed. The product may then be purified, for example, by filtering through a Celite pad and/or recrystallization, for example, from ethyl acetate and heptane.

### Method Step 6

In some embodiments of the invention, a compound of INT 5 is contacted with a protecting agent to add a protecting group on the free hydroxyl group in a compound of INT 5:

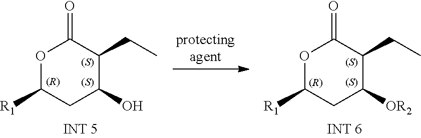

wherein R₁ is defined above and R₂ is a protecting group (e.g., THP).

In some embodiments, the protecting agent is 3,4-dihydro-2H-pyran (DHP) and it produces a tetrahydropyranyl (THP) protecting group. Other base-stable protecting groups, such as, for example, trityl or para-methoxy benzyl, which may be removed under relatively mild conditions, may be used. In some embodiments, a weakly acidic catalyst such as pyridium p-toluenesulfonate (PPTS) may be added to the compound of INT 5 that has been dissolved a solvent such as THF. The protecting agent (e.g., DHP) may then be added to the reaction flask. In some embodiments, the resulting compound of INT 6 may be redissolved in an organic solvent (e.g., MTBE) and washed.

### Method Step 7

In some embodiments of the invention, a compound of INT 6 is contacted with a hydroxide salt to open the lactone ring and form a compound of INT 7:

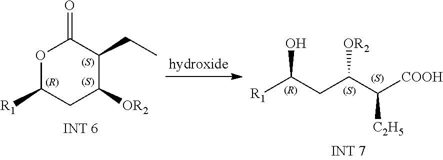

wherein R₁ and R₂ are defined above.

In some embodiments of the invention, the hydroxide salt is NaOH or KOH. In some embodiments, the compound of INT 6 is dissolved in an organic solvent (e.g., MTBE) and added to the reaction flask. An aqueous solution of the hydroxide salt (e.g., 2N NaOH) may then be added to the reaction flask and stirred. Once the reaction is complete, the aqueous base layer may be separated and the organic layer washed with a saline (e.g., 10% NaCl) solution. The compound of INT 7 may then be concentrated to form a crude oil. In some embodiments, the crude oil is azeotrope distilled with an organic solvent(s) (e.g., MTBE and THF) to further purify the compound of INT 7.

### Method Steps 8 and 9

In some embodiments of the invention, the compound of INT 7 is contacted with a protecting agent to add a protecting group to the free hydroxyl on the compound of INT 7, thus forming a compound of INT 8. The compound of INT 8 is then reacted with an acid to deprotect the protected hydroxyl group to form a free hydroxyl group, thus forming a compound of INT 9:

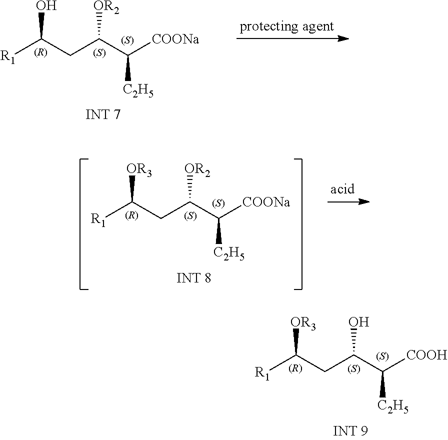

wherein R₁ and R₂ are defined above, and R₃ is an acid stable protecting group such as a benzyl or a substituted benzyl. The term “acid stable protecting group” refers to a protecting group that will not be removed by the acid added to INT 8 when forming INT 9.

In some embodiments of the invention, the intermediate product INT 8 is not purified, before forming the compound of INT 9. As such, the two reactions may be performed in a one pot synthesis. In some embodiments, the protecting agent is benzyl bromide, and it reacts with the compound of INT 7 in an O-benzylation reaction. In some embodiments, the compound of INT 7 in an organic solvent (e.g., THF) is stirred and a strong base (e.g., sodium tert-butoxide) is added, followed by the addition of benzyl bromide. Such a reaction may be performed at lower temperatures (e.g., in a range of 5° C. to 10° C.). After the reaction is complete, the reaction mixture may be heated (e.g., 50° C.). In some embodiments, an aqueous acidic solution (e.g., a mineral acid such as HCl) is added to the reaction mixture including the compound of INT8, which produces the compound of INT 9. In some embodiments, the organic and aqueous layers are separated, and the organic layer is washed and filtered and optionally further purified. In some embodiments, the product is extracted into an organic solvent such as MTBE and the organic layer evaporated to obtain the crude INT 9 reaction product. In some embodiments, the crude product may then be redissolved in an organic solvent (e.g., methyl acetate), washed with a saline solution and/or dried before filtering and taking the product to the next step. As such, in some embodiments, hydroxy THP formed from the deprotection in Method Step 9 is removed from the INT 9 product before proceeding to the next step.

### Method Step 10

In some embodiments of the invention, a compound of INT 9 is contacted wtih (S)-phenylethylamine to form a compound of INT 10:

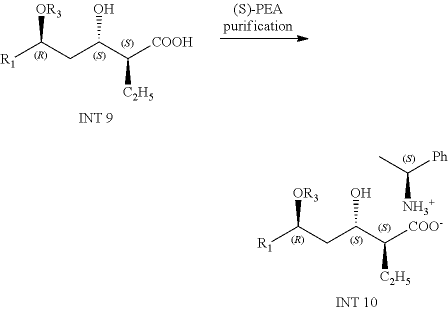

wherein R₁ and R₃ are defined above.

This optional step may be used to purify the product. In some embodiments, the (S)-PEA purification may be performed in an organic solvent, e.g., methyl acetate. In some embodiments, such a reaction occurs by dissolving the compound of INT 9 in the organic solvent such as methyl acetate. The reaction mixture may then be cooled and the (S)-(−)-α-methyl benzylamine (also referred to as (S)-(−)-1-phenylethylamine) slowly added to the reaction flask. The crystals thus formed may be filtered and washed (e.g., with cold methyl acetate).

In some embodiments, if hydroxy DHP was present in the crude mixture, the compound of INT 10 may revert to the PEA salt of INT 8 (also referred to as INT 8-PEA). As such, it may be advantageous to remove the hydroxy THP formed in Method Step 9. In cases where this reversion does occur, the INT 8-PEA can be reacted with an acid to convert the compound of INT 8-PEA to the compound of INT 9. The compound of INT 9 may then be purified by filtering, extraction, washing, and/or recrystallization (e.g., from methyl acetate). The compound of INT 9 may then be converted to the compound of INT 10 using the same (S)-PEA reaction procedure described above.

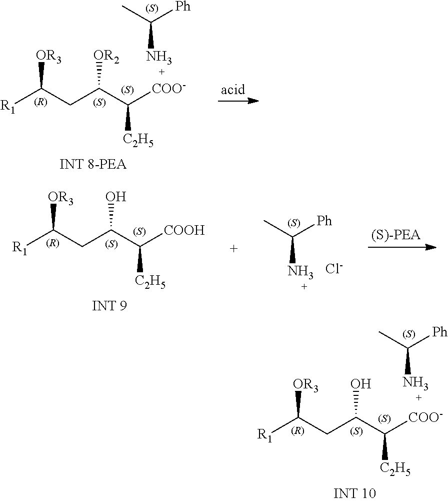

### Method Step 11

In some embodiments of the invention, a compound of INT 10 is contacted with an acid to form purified compound of INT 9:

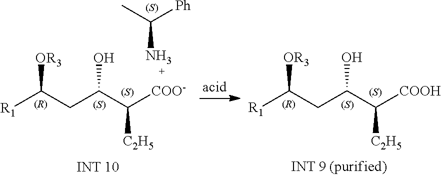

wherein R₁ and R₃ are defined above.

In some embodiments, the acid is a mineral acid such as HCl (e.g., TN HCl). In some embodiments, the compound of INT 10 is dissolved in an organic solvent such as heptane and then the aqueous acid solution (e.g., TN HCl) is added to the reaction mixture. The organic layer may then be separated from the aqueous layer, washed, dried, filtered, and/or concentrated to provide the INT 9 (purified).

### Method Step 12

In some embodiments of the invention, a compound of INT 9 or INT 9 (purified) is contacted with a dehydrating agent to form a compound of INT 11:

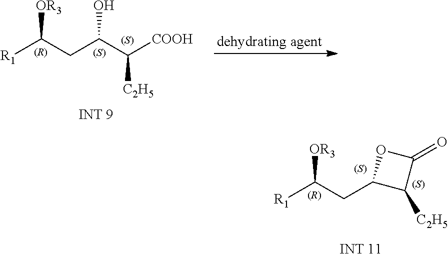

wherein R₁ and R₃ are defined above.

In some embodiments, the dehydrating agent includes one or more of benzene sulfonyl chloride, toluene sulfonyl chloride, or alkyl sulfonyl chloride, optionally in the presence of pyridine or a substituted pyridine. In some embodiments, the dehydration reaction occurs at lower temperature (e.g., less than 5° C.) and/or in an inert atmosphere. In some embodiments, the resulting INT 11 product is then extracted into heptane, washed with aqueous acidic and/or basic solutions, washed with a saline solution, dried, filtered, and and/or concentrated to produce the compound of INT 11.

### Method Step 13

In some embodiments of the invention, a compound of INT 11 is deprotected to form a free hydroxyl group and the compound of INT 12:

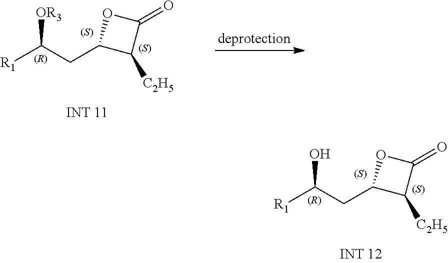

wherein R₃ is defined above.

In some embodiments. R₃ is a benzyl group and deprotection is achieved via a debenzylation reaction. In some embodiments, debenzylation is achieved using a Pd/C catalyst and hydrogen gas. In some embodiments, the Pd/C is present in the reaction mixture and hydrogen gas is bubbled through the reaction mixture under inert conditions. The resulting crude product that includes the compound of INT 12 may then be filtered and concentrated in vacuo to obtain the purified compound of INT 12.

### Method Step 14

In some embodiments of the invention, a compound of INT 12 is contacted with N-formyl L-alanine to form the compound of Formula I.

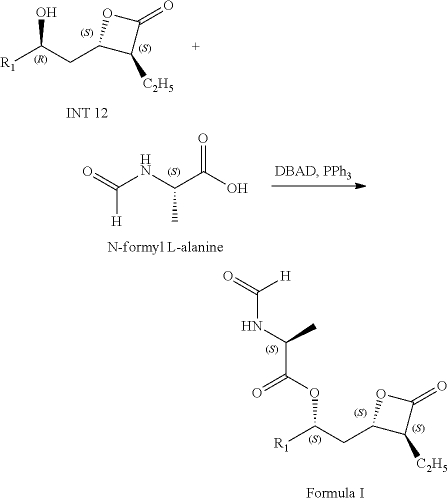

In some embodiments, a compound of 1NT 12 reacts with N-formyl L-alanine via a Mitsunobu coupling reaction using triphenylphosphine and an azodicarboxylate. Similar compounds were synthesized using diisopropyl azodicarboxylate (DIAD), but this reagent did not work sufficiently in the synthesis of the compound of Formula I. DIAD-H₂ was formed as a major side product and the compound of Formula I co-eluted with the DIAD-H₂, which made purification problematic. The inventors of the present invention surprisingly discovered that when di-tert-butyl azodicarboxylate (DBAD) was used as the azodicarboxylate, the formed DBAD-H₂ and the compound of Formula I have sufficiently different retention times and so can be separated by chromatography. The reaction also surprisingly proceeded more quickly when DBAD was used instead of DIAD. In some embodiments, the reaction of INT 12 with N-formyl L-alanine proceeds about 1.5 to about 2 times (or more) faster when in the presence of DBAD than when in the presence of DIAD.

In some embodiments, the Mitsunobu reaction is performed in an inert atmosphere. In some embodiments, the compound of INT 12, the N-formyl-L-alanine, and PPh₃ are added to a reaction flask with an organic solvent such as tetrahydrofuran (THF). The reaction may then be homogenized and cooled to a temperature within a specified temperature range that is below room temperature (e.g., to approximately 5-10° C.). In some embodiments, the DBAD is then dissolved in the same organic solvent and added slowly to the reaction flask, e.g., dropwise via an addition funnel. The DBAD may be added at a rate such that the reaction may proceed at a temperature within the specified temperature range (e.g., about 5-10° C.). Once complete, the reaction product may be concentrated and resuspended in an organic solvent such as heptane. The heptane layer may then be decanted. MTBE may then be added to the mixture and filtered to produce the crude product as an oil. In some embodiments, the crude oil is purified by column chromatography (e.g., using ethyl acetate/heptane as eluents). The combined fractions from chromatography may then be concentrated to yield an oil. In some embodiments, the crude oil is seeded with purified crytals of the compound of Formula I to produce product crystals over time. In some embodiments of the invention, the compound of Formula I is at least 90% pure (e.g., at least 90, 91, 92, 93. 94, 95, 96, 97, 98, or 99% pure, and any range defined therebetween).

Methods of the invention may further include forming a pharmaceutically acceptable salt of a compound of Formula I. The term “pharmaceutically acceptable salt” refers to a salt which retains the biological effectiveness and properties of the free base or free acid, and which are not biologically or otherwise undesirable. The salts may be formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

Methods may further include forming a composition including a compound of Formula I. In some embodiments, the compound of Formula I may be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel, Introduction to Pharmaceutical Dosage Forms, Fourth Edition 1985, 126).

In the compositions, effective concentrations of one or more compounds or pharmaceutically acceptable derivatives thereof may be mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions may be effective for delivery of an amount, upon administration, that treats, prevents, and/or ameliorates pancreatitis or inhibits lipase activity.

In one embodiment, the compositions are formulated for single dosage administration. To formulate a composition, the weight fraction of a compound of the present invention is dissolved, suspended, dispersed, or otherwise mixed in a selected carrier at an effective concentration such that the treated condition is relieved, prevented, or one or more symptoms may be ameliorated.

The active compound may be included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems and then extrapolated therefrom for dosages for humans.

The concentration of the compound of Formula I (also referred to as the “active compound”) in the pharmaceutical composition may depend on absorption, inactivation and excretion rates of the active compound, the physicochemical characteristics of the compound, the dosage schedule, and/or the amount administered as well as other factors known to those of skill in the art. For example, the amount that is delivered may be sufficient to inhibit lipase activity and/or treat pancreatitis.

The pharmaceutical compositions may be provided for administration to humans and/or animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions may, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

In some embodiments, a composition of the present invention may be suitable for oral administration. Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like may contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, gellan gum, sodium carboxymethylcellulose, polyethylene glycol 4000 and cellulose acetate phthalate.

The compound of Formula I, or pharmaceutically acceptable derivative thereof, may be provided in a composition that protects it from the acidic environment of the stomach. For example, the composition may be formulated in an enteric coating that maintains its integrity in the stomach and releases the active compound in the intestine. The composition may also be formulated in combination with an antacid or other such ingredient. When the dosage unit form is a capsule, it may contain, in addition to material of the above type, a liquid carrier such as a fatty oil. In addition, dosage unit forms may contain various other materials which modify the physical form of the dosage unit, for example, coatings of sugar and other enteric agents. The compounds may be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials may also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives.

Pharmaceutically acceptable substances used in non-effervescent granules, to be reconstituted into a liquid oral dosage form, include diluents, sweeteners and wefting agents. Pharmaceutically acceptable substances used in effervescent granules, to be reconstituted into a liquid oral dosage form, include organic acids and a source of carbon dioxide. Coloring and flavoring agents are used in all of the above dosage forms. Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, xanthan gum, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation. For a solid dosage form, the solution or suspension, in for example propylene carbonate, vegetable oils or triglycerides, is in one embodiment encapsulated in a gelatin capsule. For a liquid dosage form, the solution may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(loweralkyl) acetals of loweralkyl aldehydes such as acetaldehyde diethyl acetal.

Parenteral administration of the compositions includes intravenous, subcutaneous and intramuscular administrations. Administration can be intraperitoneal or directly into or near an organ or tissue of interest, e.g., pancreas. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, xanthan gum, hydroxypropyl methylcellulose and polyvinylpyrrolidone. Emulsifying agents include Polysorbate 80 (TWEEN™ 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The compound of Formula I may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized powders, which can be reconstituted for administration as solutions, emulsions and other mixtures, may also be used to carry out the present invention. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Also provided according to embodiments of the invention are compounds of Formula I formed by a method of the invention. Further provided are pharmaceutically acceptable salts of compound of Formula I formed by a method of the invention. Further provided are compositions including a compound of Formula I prepared by a method of the invention and, optionally, a pharmaceutically acceptable carrier.

A compound, a pharmaceutically acceptable salt, and/or a composition of the invention may also be used to inhibit lipase activity in a subject and/or treat pancreatitis. The term “subject” includes both humans and animals.

The present invention is explained in greater detail in the following non-limiting experimental section.

### EXAMPLES

The labeling/numbering of compounds provided in the examples sections is relevant to the examples section only and may not correspond to the labeling/numbering provided throughout the rest of the present application. Thus, the labeling/numbering of compounds in the examples section is not to be confused with the labeling/numbering of compounds throughout the rest of the application (e.g., in the summary and detailed description sections and claims).

Abbreviations may include: round bottom flask (RBF), starting material (SM), room temperature (RT), dichloromethane (DCM, or CH₂Cl₂), ethyl acetate (EtOAc), hexanes (hex), methanol (MeOH), isopropanol (IPA), diethyl ether (Et₂O), acetic acid (AcOH), 1-2-dichloroethane (1,2-DCE), tetrahydrofuran (THF), dimethylformamide (DMF), cesium carbonate (Cs₂CO₃), sodium sulfate (Na₂SO₄), and silica (SiO₂).

### Example 1: Preparation of INT 1

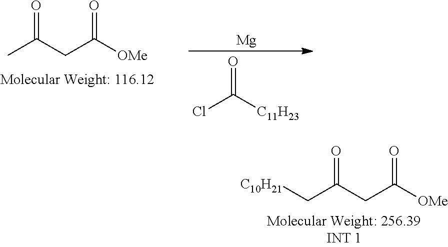

A 30 L jacketed vessel was equipped with a vacuum rated stirrer bearing, argon gas inlet and outlet, connected heater/chiller unit and a thermocouple/controller. The vessel was purged with argon for 15 minutes. Methanol (3.85 L) was added to the vessel followed by magnesium (120 g, ˜50 mesh). The sides of the vessel were rinsed with methanol and the vessel was heated to 55° C. and then to 65° C. The reaction was stirred at 65° C. overnight. Toluene (11.6 L) was added to the vessel and a distillation head was attached to the vessel. The temperature of the circulation solution was increased to 100° C. and distilled ˜1.5 L solvent. The circulation solution was decreased to 45° C., and methyl acetoacetate (2.2 Kg) was added to the vessel at 45° C. During the addition, the temperature increased by 10° C. The reaction mixture was stirred at 45° C. for 12 h. The temperature of the circulation solution was increased gradually to 140° C. and distilled ˜4.9 L solvent. The temperature on the chiller was set to 60° C. Lauroyl chloride (1608 g) was dissolved in toluene (1.55 L). The solution was transferred to an addition funnel and added to the 30 L vessel over 2-3 h. The reaction mixture was stirred at 60° C. for 12 h.

### 1. Quench

Methanol (1.8 L) was added to the reaction mixture. The color of the reaction mixture changed from orange to red and all the solid was dissolved. The reaction mixture was distilled ˜900 ml using a distillation head and vacuum pump. The reaction mixture was stirred at 75° C. until the majority of tricarbonyl intermediate converts to product on TLC. The reaction mixture was cooled to 25° C. and conc. HCl (1502 g) was added. The reaction mixture turned from an orange to yellow color.

### 2. Work-up & Isolation

The reaction was stopped and the HCl layer was separated. The organic layer was washed with water (2×3.5 L), 2% KHCO₃ (1×2.8 L) and water (1×2.8 L). The organic layer was dried over anhydrous Na₂SO₄ (˜300 g) for 1 h. The organic layer was filtered and concentrated in vacuo to yield orange oil (2018 g, >100%).

### 3. Crystallization

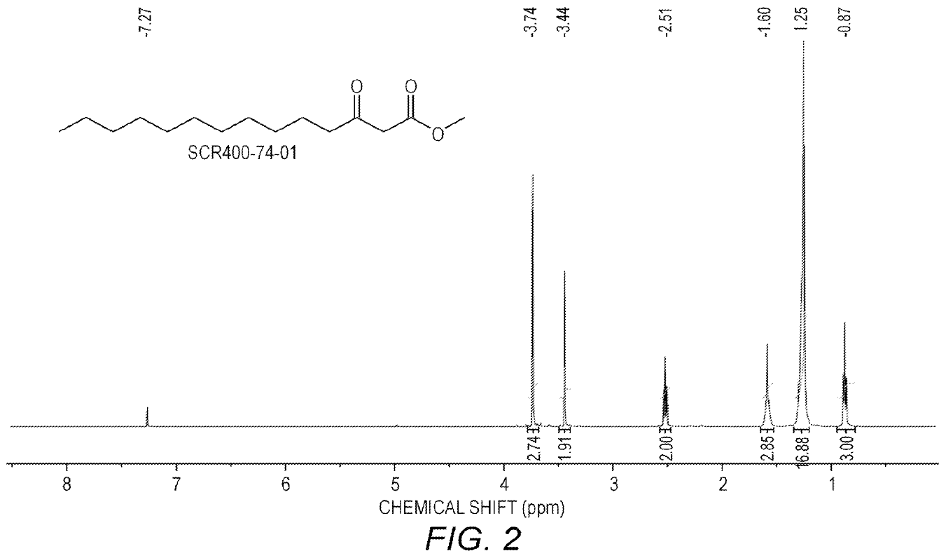

The crude was dissolved in methanol (6054 mL) and cooled in the freezer to crystallize. The crystals were filtered through a P3 frit funnel and air-dried for 2 h. The solid was transferred into trays and drying continued in an oven at RT until constant mass. INT 1 was isolated as a flaky white to off white solid (1248 g, 66%). The ¹H NMR Spectra of INT 1 is shown in FIG. 2.

### Example 2: Preparation of INT 2

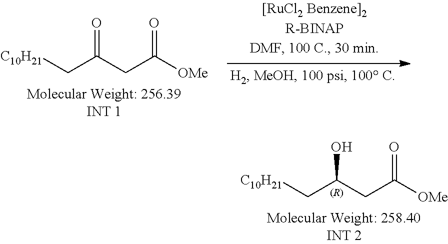

Enantioselectivity in this reaction was found to depend on several factors, including solvent, temperature, and pressure. In particular, R&D experiments established that the ratio of reaction volume to reactor dead space was a key parameter in lower pressure hydrogenations (<1000 psi). For a 2 L reactor volume, the reaction scale was fixed at 150 g of INT 1 (at 200 psi) or less to avoid the hydrogen-starved conditions associated with reduced enantioselectivity.

### 1. Enantioselective Synthesis of INT 2

DMF and methanol were degassed for 30 minutes with argon. In a 3-neck flask, evacuated and purged with argon 3 times, added [RuCl₂benzene]₂ (1.95 g, 3.89 mmol, Sigma), R-BINAP (2.7 g, 4.33 mmol, Sigma) and degassed DMF (95 mL, Sigma). The reaction mixture was heated to 100° C. and stirred for 30 minutes and then cooled to RT.

INT 1 (150 g, 1.1 mol) was dissolved in degassed methanol (325 mL). The mixture was transferred into a 2 L autoclave and degassed with argon for 15 minutes. The above prepared active catalyst was transferred into the autoclave with argon purging. The autoclave was evacuated, purged with hydrogen 3 times and filled with hydrogen (200 psi). The reaction was heated to 100° C. and stirred for 24 h.

### 2. Work-up & Isolation

TLC and LCMS of the reaction mixture suggested completion of reaction. The reaction mixture was filtered through a celite bed and the bed was washed with ethyl acetate (100 mL). The filtrate was concentrated in vacuo to yield an oil.

The crude oil was dissolved in ethyl acetate (10 vol) and added (10 vol) of water. The organic layer was separated and washed with saturated NaCl (2×). The organic layer was dried over anhydrous Na₂SO₄ for 1 h. The organic layer was filtered and concentrated in vacuo to yield an off white solid. The reaction to prepare INT 2 was repeated through seven iterations.

### 3. Crystallization

The crude (960 g) was taken into a round bottom flask and heptane (5 vol) was added. The mixture was heated to 65-70° C. to form a homogeneous solution. The solution was cooled to RT and then 0-5° C. The flask was kept in the refrigerator overnight to form more crystals. The crystals were collected on a coarse frit funnel, then air and vacuum dried for 3 h to yield off white solid (870 g, 90% recovery).

### Example 3: Derivatization of INT 2

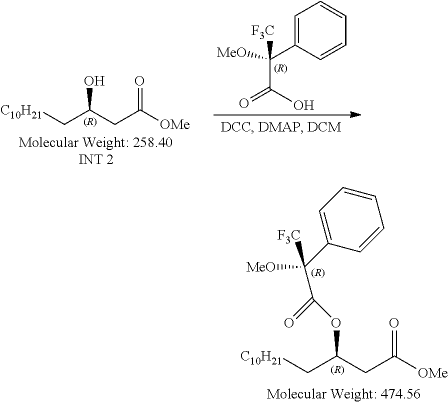

In a 10-dram vial, INT 2 (10 mg), Mosher's acid (9.1 mg), DMAP (0.004 mg), DCC (12 mg), DCM (1 mL) and magnetic stir bar were added. The homogeneous solution was stirred at RT overnight. The reaction mixture was filtered, and the mother liquor was evaporated. The crude was submitted for ¹H NMR.

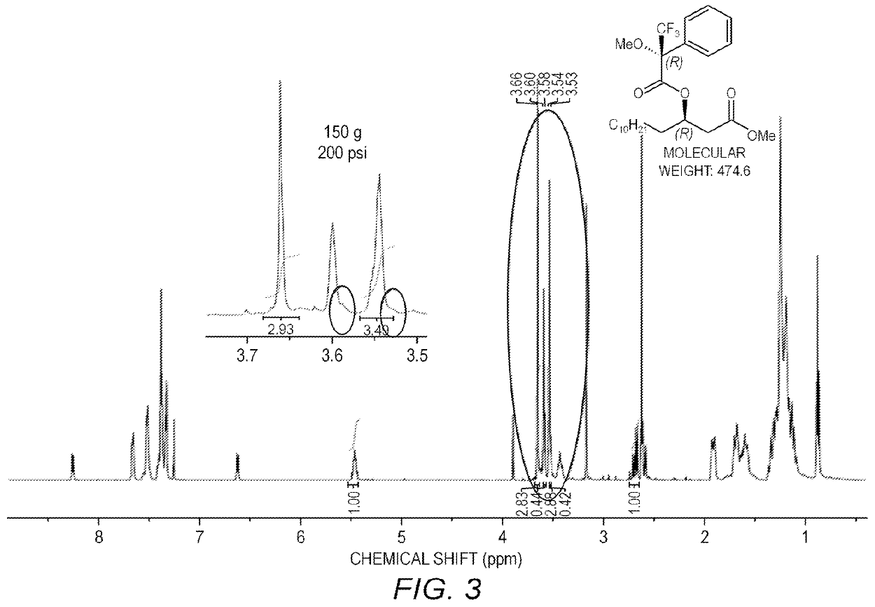

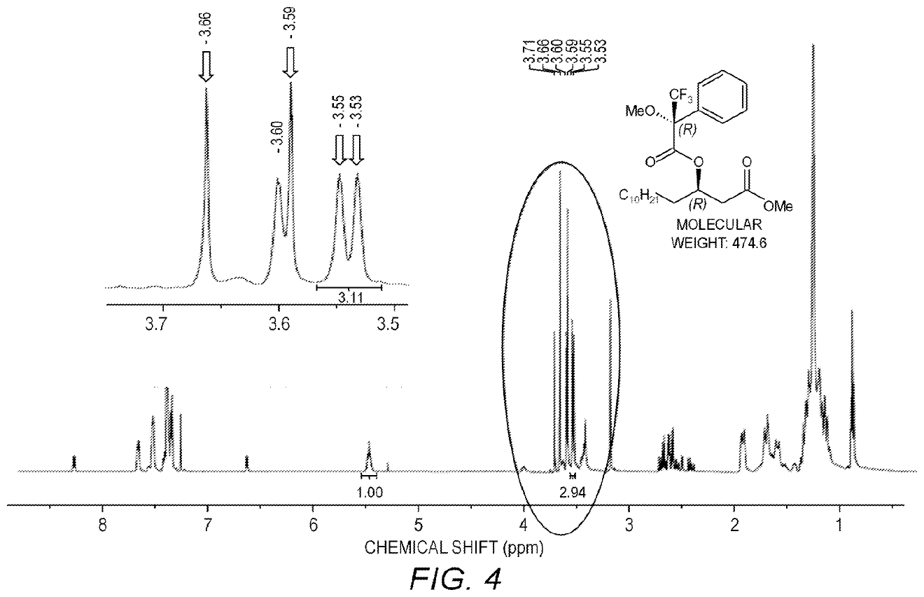

Referring to FIG. 3, the presence of two methyl singlets between 3.5 to 3.7 suggested that INT 2 is enantiomerically enriched (racemic mixture shows two sets of two singlets, see FIG. 4). A chiral HPLC method was also developed to measure enantiomeric excess (ee).

### Example 4: Derivatization of INT 2

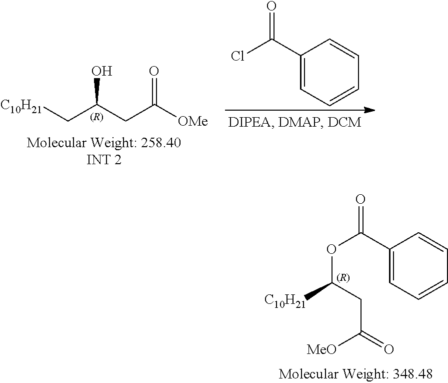

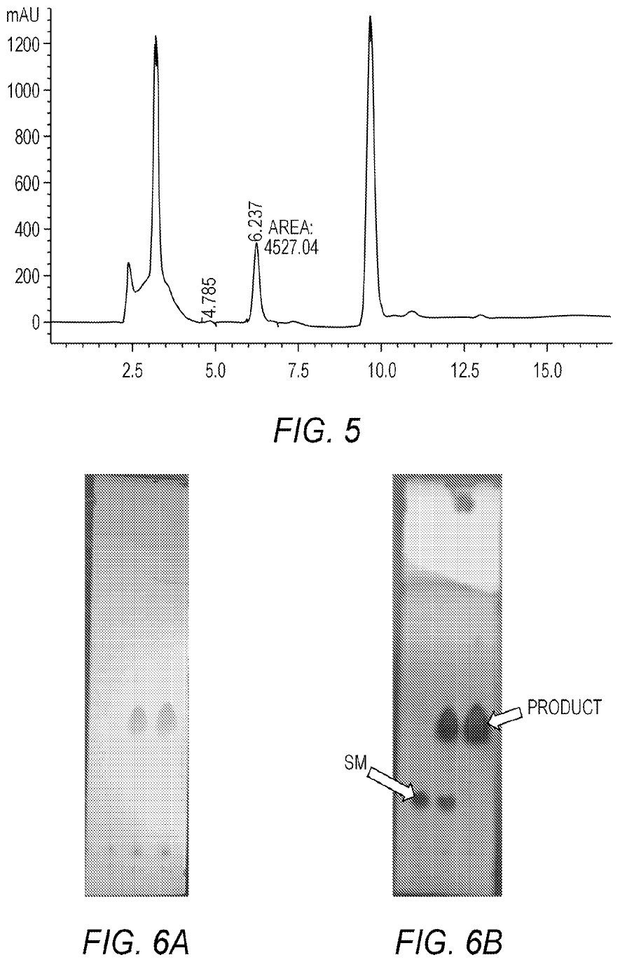

In a 10-dram vial, INT 2 (50 mg), DMAP (2.3 mg, Aldrich), DIPEA (37.5 mg), benzoyl chloride (54.4 mg, Aldrich), DCM (0.5 mL) and a magnetic stir bar were added. The vial was stirred at RT for 16 h. An aliquot (10 μL) was dissolved in 10% HPLC grade isopropyl alcohol in hexane (1 mL, isopropyl alcohol; hexane) and submitted for chiral HPLC. The chiral HPLC data (FIG. 5) suggests that the batch was enantiomerically enriched and the percentage of enantiomeric excess (% ee) was 97%.

### Example 5: Preparation of INT 3

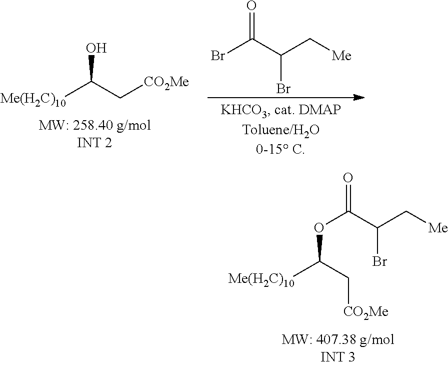

A 3-neck 12 L RBF was equipped with a cooling bath with coils, thermocouple/controller and overhead stirrer. INT 2 (360 g, 1.39 mol, 1 eq), DMAP (17 g, 0.139 mol, 10 mol %) and toluene (720 mL, 2 vol) were charged resulting in an endotherm (T: 17° C. to 4.6° C.). All solids were dissolved once the temperature warmed to ˜13° C. The chiller was set to 4° C. and propylene glycol was charged to the cooling bath. Dry ice was used to quickly cool the bath to 10-15° C. and the chiller was used to maintain that temperature. KHCO₃ (474 g, 4.74 mol, 3.4 eq) and water (176 mL, 9.75 mol, 7 eq) were charged to the RBF. Once the mixture cooled to 15-10° C., the first portion of acid bromide (255 mL, 2.09 mol, 1.5 eq, density: 1.88 g/mL, Oakwood) was charged via addition funnel over 3 h. CO₂ began to evolve once the acid bromide was charged and stopped ˜30 min after the addition was complete. TLC indicated that the reaction was incomplete with 10-30% starting material remaining. The second charge of acid bromide (130 mL, 1.05 mol, 0.75 eq, Oakwood) was charged to the addition funnel and was adjusted to slowly drip overnight at 10-15° C.

### 1. Work-up & Isolation

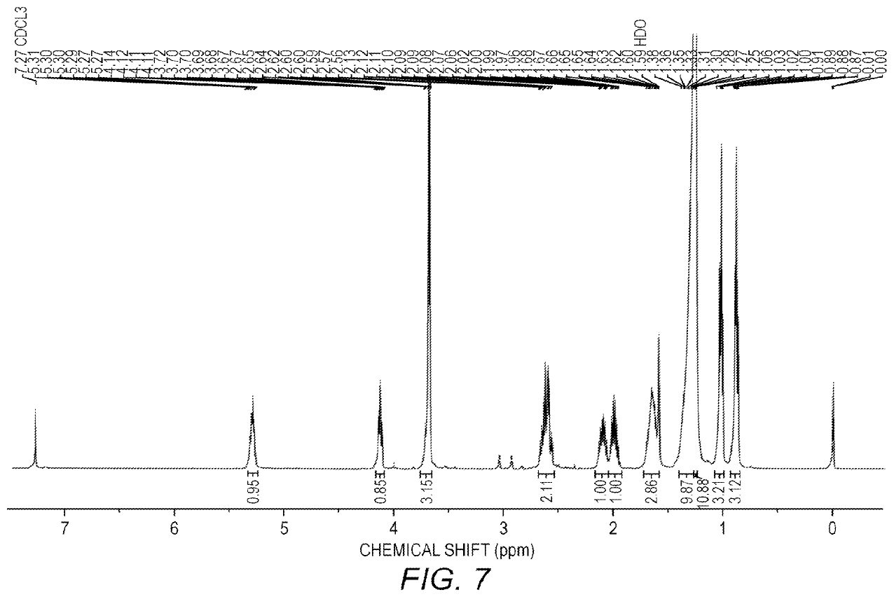

The next morning there was ˜10 mL of acid bromide remaining in the addition funnel. The remainder of reagent was released to the reaction and TLC and LC/MS (22 h) revealed that the reaction was complete. The cooling bath was replaced with a ˜40° C. water bath. Water (1.8 L, 5 vol) and MTBE (1.8 L, 5 vol, Aldrich) were charged to the reaction mixture, causing the temperature to rise to 25-30° C. It was then stirred for 30 min to hydrolyze any unreacted acid bromide. Stirring was stopped and once the layers separated into phases the pH was ˜8. The water bath was removed, and the aqueous phase siphoned from the 12 L RBF. The organic phase was washed with water (2×, 720 mL, 2 vol) until the pH of the washings was pH 7. The organic phase was transferred to a tared 5 L RBF (any remaining water in the 12 L RBF was removed using a 500 mL separatory funnel) and concentrated in vacuo at 30 to 60° C. There was sediment suspended in the product, and it was diluted with toluene (200 mL) and polished filtered with a tared coarse frit (˜800 mg of wet solid remained) into a tared 3 L round bottom flask. The flask and frit were then rinsed with minimal toluene. The solvent was again removed in vacuo at 60° C. for 2-3 h to yield INT 3 as a dark brown oil (590.76 g, 104% yield). FIG. 6A shows the TLC of INT 3 under UV lamp and FIG. 6B shows the TLC under PMA stain. The eluent was 20% ethyl acetate/heptane. FIG. 7 is the ¹H NMR of crude INT 3.

### Example 6: Preparation of INT 4

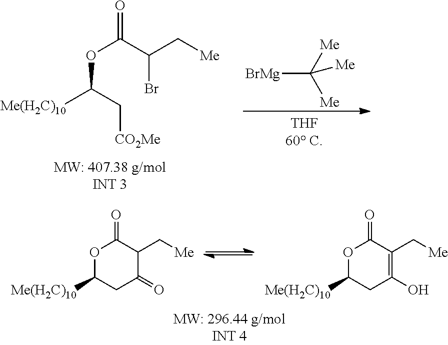

A 4-neck 22 L RBF was equipped with an overhead stirrer, heating mantle, two 1 L addition funnels with gas inlets on each and a thermocouple/controller. Minimal THF (1000 mL, 1.75 vol) was charged to the RBF so that solvent contacted the thermocouple. The controller was set to 60° C. and argon flow was initiated. After 20-30 min, INT 3 (570 g, 520 mL, 1.40 mol, 1 eq) was warmed to ˜40° C. to reduce its viscosity and was charged to a 1 L funnel. 1M tert-butyl Grignard (4.8 L, 4.9 eq, 3.5 eq) was charged in 800 mL bottle increments by removing the Sure Seal cap and pouring it into the argon purged addition funnel.

Once the temperature reached 55-60° C., ˜20 mL of Grignard reagent was charged to the hot THF to deoxygenate and dehydrate the solvent. The reagent solutions were continuously charged to the hot THF simultaneously over 4 h (800 mL of Grignard and ˜85 mL of INT 3 was dispensed every 40 min) with vigorous isobutylene formation. Once the addition was complete, the reaction mixture was stirred at 60° C. TLC (6.5 h) indicated that INT 3 was consumed, however the reaction was allowed to age until 8 h mark and heating was turned off.

### 1. Work-up

The following morning LCMS confirmed that SM was consumed. The reflux condenser was replaced with a distillation head and the temperature was set to 75° C. Solvent was distilled until 2.3 L of THF (46%, 4 vol) was collected leaving ˜2.7 L (4.7 vol) in the still pot. The heating mantle was replaced with an ice bath and the reaction vessel was then cooled to 0-10° C. Cold (10° C.) 0.24M citric acid (9 L, 1 mol, 1.4 eq, 14 vol) was charged over 20 min (T: 8-40° C.) the addition of the first 500 mL was exothermic. The product began to precipitate, and the mixture was stirred for 1.5 h, after which the large particles were broken up to make the solid easier to filter. The solid was filtered with a tared coarse 3 L frit and washed with water (8×1 L, 1.8 vol.) until the washings were pH 6-7. The filter cake was dried under vacuum for 72 h, yielding the crude product as a brown solid (642.92 g, 155% yield) wetted with water. The wet cake was suspended in toluene (2 L, 3 vol) in a 5 L RBF and the resultant mixture then distilled in vacuo at 50-70° C. The distillation was repeated twice after charging with toluene (300 mL). A total of 180 mL of water was collected in the distillate.

### 2. Crystallization

The solids in the evaporation flask were transferred to a 22 L 4-neck RBF equipped with a heating mantle, thermocouple/controller, a reflux condenser and overhead stirring using warm toluene (3×500 mL; Total: 1.5 L, 2.7 vol.). The mixture was heated to 80° C. and heptane (3 L, 5.3 vol) was charged at a rate such that the temperature was maintained at 70-80° C. Once the addition was complete, heating was turned off and the mixture was allowed to crystallize as it cooled to ambient temperature overnight.

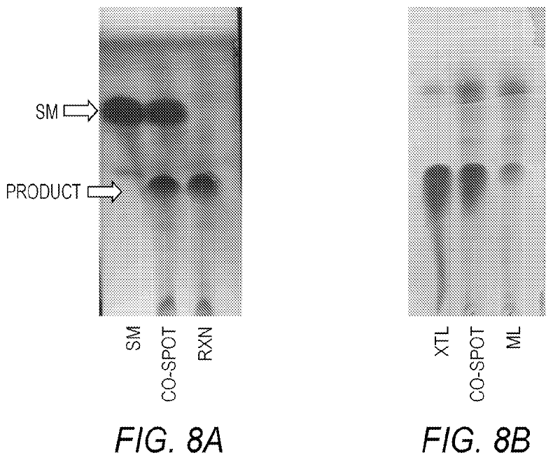

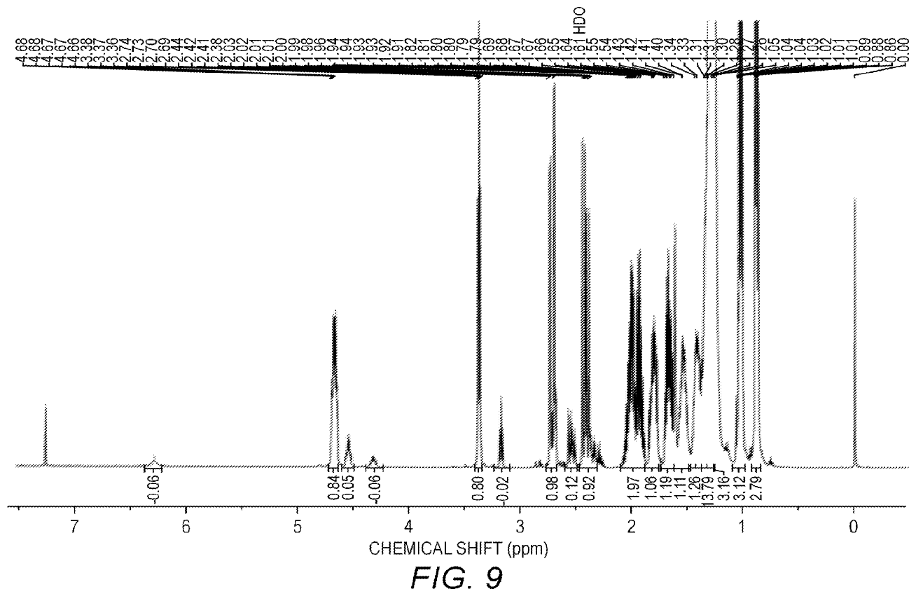

The following morning, the resulting slurry was filtered with a tared coarse 3 L frit and washed with 30% toluene/heptane (3×666 mL) and dried for 5-10 min. It was then placed in a 55° C. oven until constant mass to yield an off-white solid (269.5 g, 65% yield). FIGS. 8A and 8B show the TLC of INT 4 (FIG. 8A—crude product (IPC check); FIG. 8B—crystallized product). The eluent was 50% ethyl acetate/heptane. A PMA stain was used. ¹H NMR of INT 4 (keto-enol tautomerism) is shown in FIG. 9.

### Example 7: Preparation of INT 5

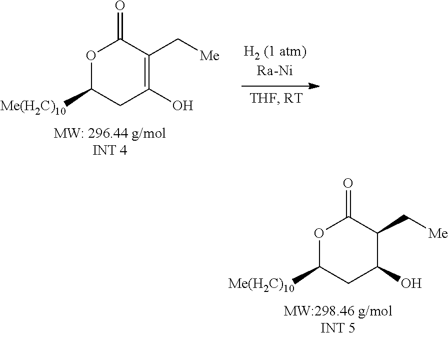

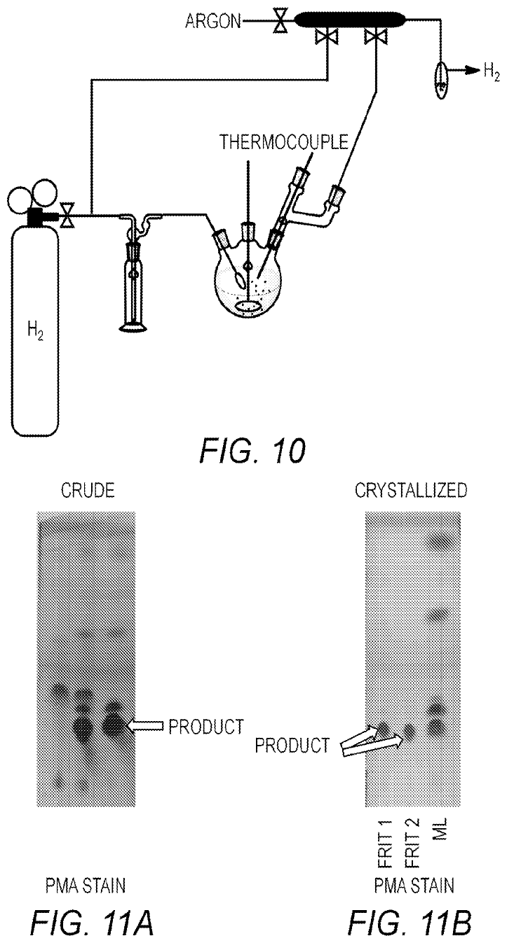

Freshly prepared Raney-Ni (˜700 g, 150 wt %) was stirred in THF (12.9 L, 20 vol.) in a 50 L three neck RBF equipped with a thermocouple, pneumatic overhead stirrer, gas diffusion sparger under argon. See FIG. 10 for reaction configuration. INT 4 (648 g, 2.18 mol, 1 eq.) was charged and hydrogen was passed through the system directly through the reaction mixture via gas diffusion sparger. The reaction was then stirred vigorously overnight.

After 18 h of stirring, the hydrogen flow was stopped and then argon was passed through the system for 5-10 min before the reaction was opened to atmosphere. Samples were then removed for ion pair chromatography (TLC & LCMS). LCMS did not detect any starting material, but TLC indicated that there was starting material remaining. It was noted that product began to crystallize in the sparger, it was partially dissolved with 3-4 THF rinses resulting in better gas flow. The reaction was then restarted by re-initiation of hydrogen flow and stirring. It was stirred again for another 24 h. Both TLC and LCMS indicated completion at the 18 h mark and the reaction was deemed complete.

### 1. Celite Pad Preparation

A 1-2 cm layer of sand was charged to a 2 L coarse frit and smoothed over. A slurry of 100 wt % Celite (500 g, AW standard Super-Cel NF; Sigma Aldrich) in minimal THF was separately prepared. A piece of filter paper was placed on top of the sand and the slurry was charged on top of the filter paper so that it formed a 1-2 cm layer of Celite. The Celite was allowed to settle, then light vacuum was applied to form a compact Celite pad.

### 2. Ra—Ni Removal & Product Isolation

Once the reaction was complete, stirring was stopped and Ra—Ni was allowed to settle to the bottom of the round bottom flask. The supernatant was transferred via vacuum siphon to a 4 L vacuum flask so that a minimum of Ra—Ni was removed. The flask was then poured onto the Celite pad and filtered. Any residual Ra—Ni that was filtered was wetted with THF at all times. THF (4 L) was charged to the reaction round bottom flask containing the spent Ra—Ni then, stirred and allowed to settle. It was siphoned as before, then filtered through Celite and this process was repeated until no product was detected (TLC) in the supernatant. THF rinses (2×2 L). The product solution was concentrated in vacuo using the rotovap yielding crude product as a white solid (769 g).

### 3. Crystallization

A 12 L three-neck RBF was equipped with an overhead stirrer, thermocouple, and heating mantle. The solids from the 20 L evaporation flask were charged to the 12 L RBF. The residual solids in the 20 L evaporation flask were removed with EtOAc (3250 mL, 5 vol) and charged to the crystallization flask. Stirring was initiated and it was heated to 75° C., and the solids dissolved at 60-70° C. Heptane (7800 mL, 12 vol) was charged in portions so that the temperature was 70-80° C. Heating was then turned off and the mixture was allowed to cool to ambient temperature over the weekend.

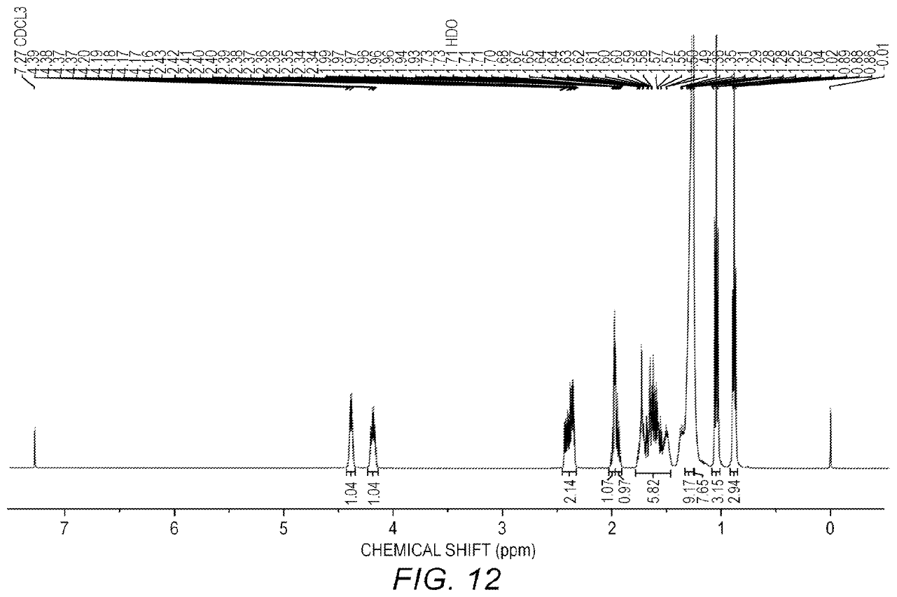

The resulting slurry was filtered with a tared coarse frit and the wet cake was washed with heptane (1000 mL) then dried for 15-30 min under vacuum. It was then placed in a 35° C. vacuum oven until constant mass giving INT 5 as an off white solid (453 g, 69% yield). FIG. 11A and FIG. 11B show the TLC of crude INT 5 (FIG. 11A) and crystallized INT 5 (FIG. 11B). FIG. 12 shows the ¹H NMR of crystalline INT 5.

### Example 8: Preparation of INT 6

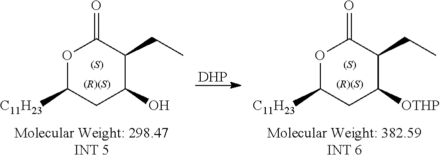

A 3-neck 12 L RBF was equipped with an overhead stirrer, heating mantle, argon gas in and outlets and a thermocouple/controller. INT 5 (452 g, 1.5 mol) was added to the flask followed by THF (4.5 L, 10V) and stirred at RT. The solid was partially dissolved at RT. Pyridinium p-toluenesulfonate (5.7 g, 0.015 eq., 0.023 mol) was added to the flask. 3,4-Dihydro-2H-pyran (382.2 g, 3 eq., 4.54 mol, Aldrich) was added dropwise to the reaction mixture over 1 h. The reflux condenser was set at 8° C. and the reaction mixture was heated at 50° C. for 24 h.

### 1. Work-up & Isolation

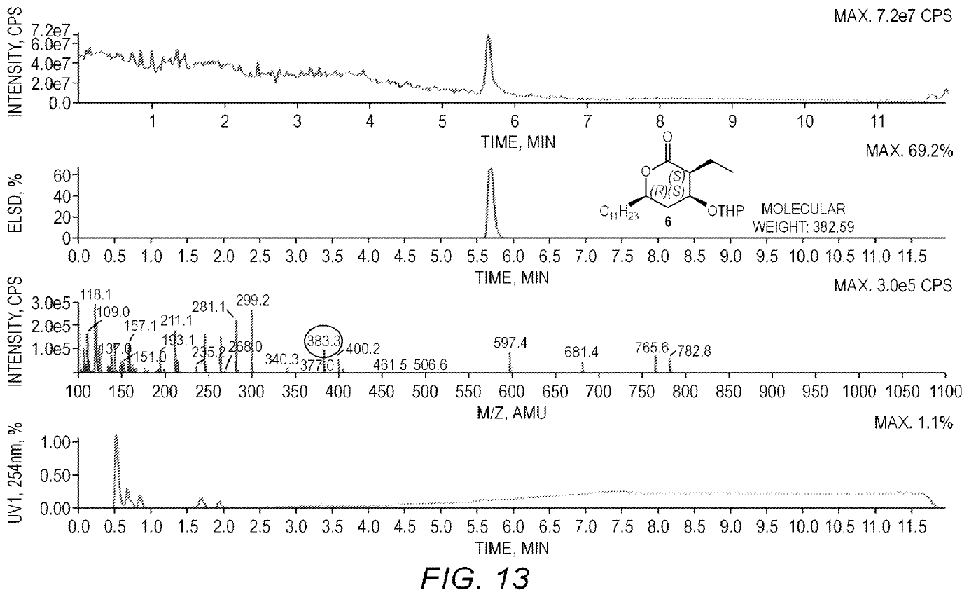

TLC & LCMS confirmed the reaction was complete (See FIG. 13). The reaction mixture was transferred into a 22 L rotavap flask and concentrated in vacuo. The resultant crude oil was dissolved in MTBE (4 L) and the organic layer was washed with water (3×2 L), followed by saturated NaCl (1×2 L). The organic layer contains traces of water, which was taken forward for the next step.

### Example 9: Preparation of INT 7

A 3-neck 12 L RBF was equipped with an overhead stirrer, cooling bath and a thermocouple/controller. INT 6 (579 g. 1.5 mol) which was dissolved in MTBE (4.6 L, 8V) from the previous step was added to the flask. 2N NaOH solution was prepared by using 32% NaOH solution. 2N NaOH (1.3 L, 2.25 eq.) was added to the reaction mixture and stirred vigorously at RT for 20 h.

### 1. Work-up & Isolation

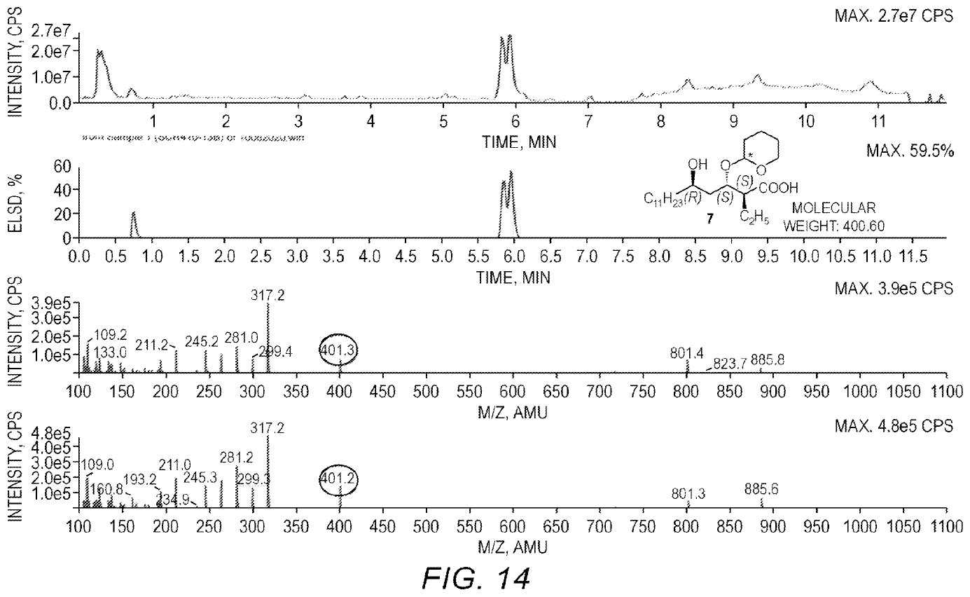

LCMS confirmed the reaction was completed (See FIG. 14) The reaction was stopped and the 2N NaOH layer was separated. The organic layer was washed with 10% NaCl (3×2 L). The organic layer was dried over anhydrous Na₂SO₄ (˜300 g) for 1 h. The organic layer was filtered and concentrated in vacuo to yield an oil. The crude oil was azeotrope distilled with MTBE (2×2 L) and THF (2×2 L). At the end of evaporation all the oil turned into mixture of solid lumps and powder. The crude mixture was kept under vacuum overnight for further drying (694 g, >100%).

### Example 10: Preparation of INT 9

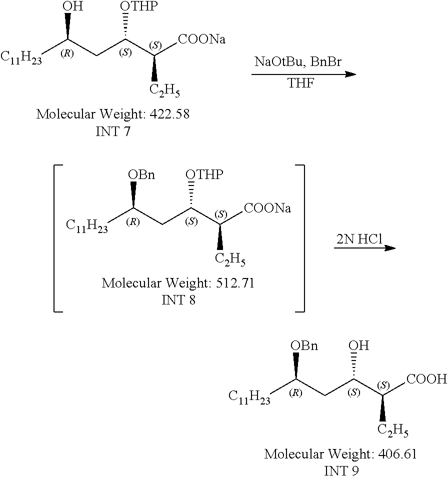

A 3-neck 22 L RBF was equipped with an overhead stirrer, cooling bath, argon inlet & outlet and a thermocouple/controller. INT 7 (639 g; 1.5 mol; big chunks were broken into small pieces) and powder was added to the flask. THF (6.3 L, TOV) was added to the flask and stirred vigorously at RT. The slurry mixture was cooled to 5-10° C. Sodium tert-butoxide (290.6 g, 3.0 mol, 2 eq) was added portion-wise to the reaction mixture over 1 h. The reaction mixture was stirred at 5-10° C. for 1 h 30 minutes. During the stirring period the slurry became cloudy, and the color of the reaction mixture changed from light orange to dark orange. Benzyl bromide (388 g, 270 mL, 1.5 eq, 2.3 mol) was diluted with THF (250 mL) and added to the reaction mixture dropwise over 1 h while maintaining the temperature at 5-10° C. The ice bath was removed, and the reaction mixture was stirred at RT for 20 h.

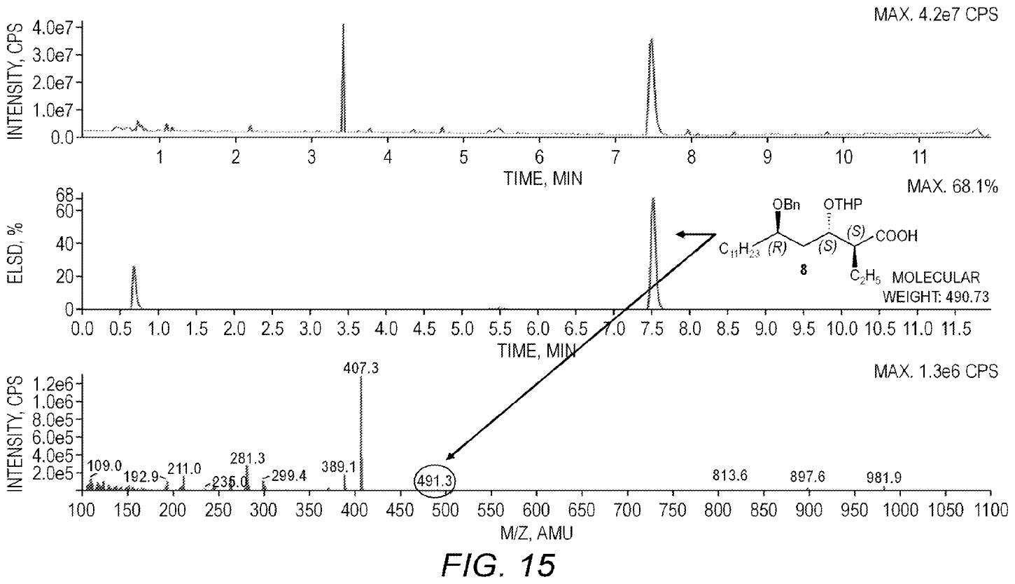

LCMS data suggested that 50% of starting material remained and the reaction had ceased. A small aliquot was removed to test run the reaction at 50° C. After 1 h, the test run LC-MS data suggested that the reaction was complete. The cooling bath was replaced with a heating mantle for the reaction and heated at 50° C. over the weekend. Subsequent LCMS data suggested that the reaction was complete. The LC-MS of INT 8 is shown in FIG. 15.

Heating was stopped and the reaction mixture stirred while cooling to room temperature. 2N HCl (2.5 L, 4V) was added to the pot over 1 h, maintaining the temperature below 45° C. The slurry reaction mixture turned into a clear solution. The reaction mixture was heated at 50° C. for 5 h. Heating was removed and the resultant mixture was stirred overnight to cool.

### 1. Work-up & Isolation

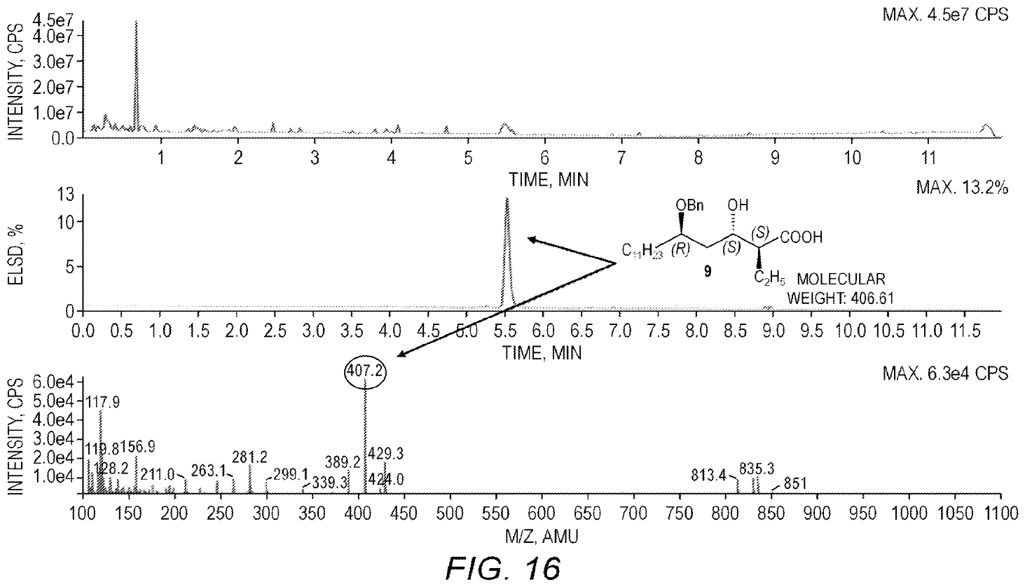

LCMS confirmed the reaction was complete (See FIG. 16). MTBE (5 L) was added to the reaction and stirred for 15 minutes. The stirring was stopped to settle the layers and the 2N HCl layer was separated. The organic layer was washed with saturated NaHCO₃ (4×4 L) to pH 8-9. The organic layer was dried over anhydrous Na₂SO₄ (˜300 g) for 1 h. The organic layer was filtered and concentrated in vacuo to yield a thick brick red oil (783 g, >100%, contains traces of solvent). The crude oil was redissolved in ethyl acetate (4 L). The organic layer was washed with 0.5N HCl (1 L), followed by water (2×2 L), brine (1×2 L). The organic layer was dried over anhydrous Na₂SO₄ (˜200 g) for 1 h. The organic layer was filtered and concentrated in vacuo to yield thick brick red oil (765 g, >100%, contains traces of solvent).

To avoid the reverse reaction to INT 8-PEA, the work-up procedure may be modified. Once the reaction with 2N HCl finishes, the product may be extracted into MTBE and the organic layer evaporated to obtain crude product. The crude may then be re-dissolved in methyl acetate and washed with water, brine and dry over Na₂SO₄. The organic layer (methyl acetate) may be filtered and taken forward to next step (INT 10).

### Example 11: Preparation of INT 10

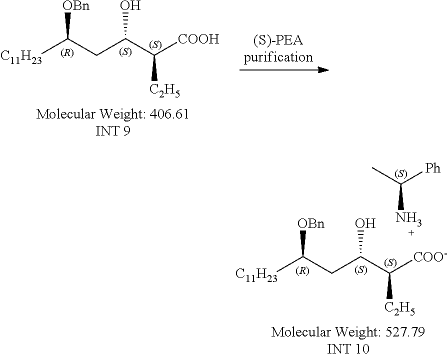

A 3-neck 12 L RBF was equipped with an overhead stirrer, cooling bath, argon inlet and outlet, and a thermocouple/controller. INT 9 (615 g, 1.5 mol) in a 22 L rotavap flask was dissolved in methyl acetate (3.7 L, 6V) and transferred into a 12 L RBF. The reaction mixture was stirred and cooled to 5-10° C. (S)-(−)-alpha methylbenzylamine ((−) PEA, 183.3 g, 195 mL, Chem-Impex) was charged to the addition funnel and added to the reaction mixture dropwise maintaining the temperature at 5-10° C. The thick solid was stirred at RT for 16 h.

### 1. Crystallization & Isolation

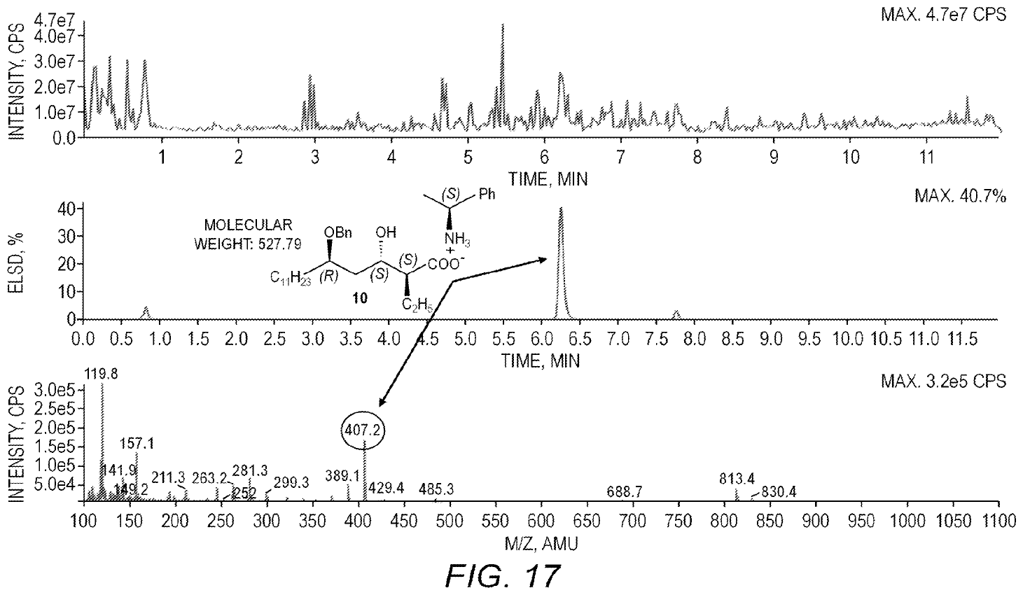

The resultant crystals were cooled to 5-10° C. and stirred for 2 h. The crystals were collected on a coarse frit funnel and the solids were washed with cold methyl acetate (1 L). The crystals were air dried for 1 h and then vacuum oven dried at RT until constant weight. INT 10 was isolated as light yellow solid (472.7 g). The LS-MS spectra of INT 10 is shown in FIG. 17.

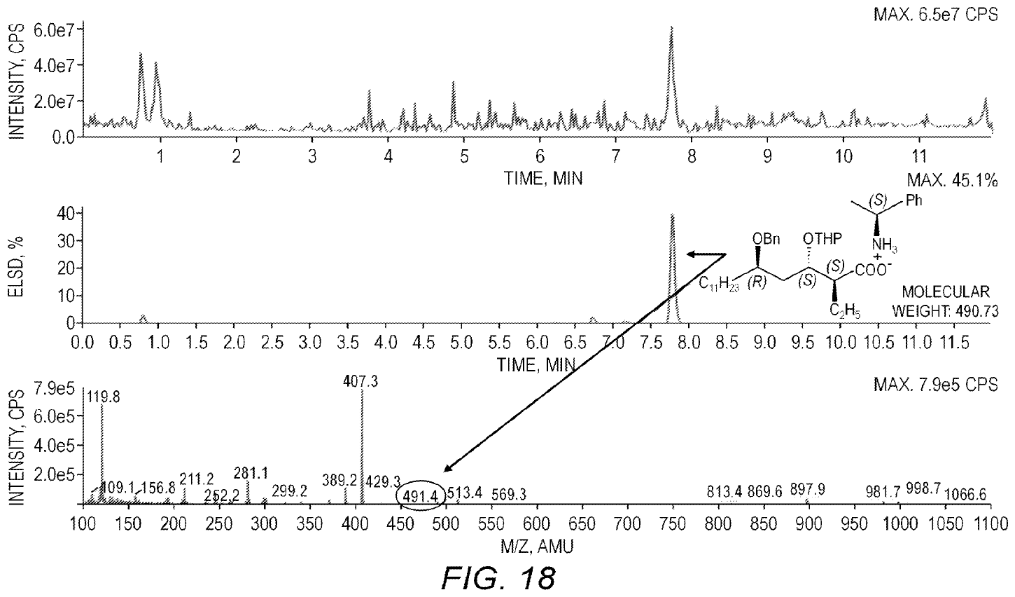

LCMS data of the mother liquor (FIG. 18) showed that some of the INT 10 had reverted to the PEA salt of INT 8 (INT 8-PEA). This is probably due to the presence of DHP in the crude. A test reaction was performed with INT 8-PEA to convert it to INT 9 and then to INT 10 as shown in below scheme. The test reaction was successful, and the remaining material was converted from INT 8-PEA to INT 10 by this method.

The conversion of the mother liquor (INT 8-PEA) to INT 10 is shown below.

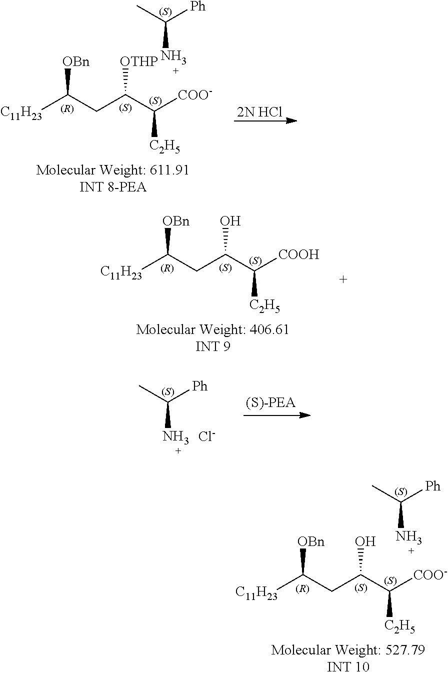

2. INT 8-PEA to INT 10 (from Mother Liquor)

A 3-neck 12 L RBF was equipped with an overhead stirrer, heating mantle, and a thermocouple/controller. INT 8-PEA (SCR410-18B, 480 g, 0.7 mol) in a 22 L rotavap flask was dissolved in MTBE (2.4 L, 5V) and transferred into a 12 L RBF. 2N HCl (2.5 L, 4V) was added to the pot and the reaction mixture was heated at 50° C. for 16 h. Heating was removed and the mixture stirred while cooling to RT.

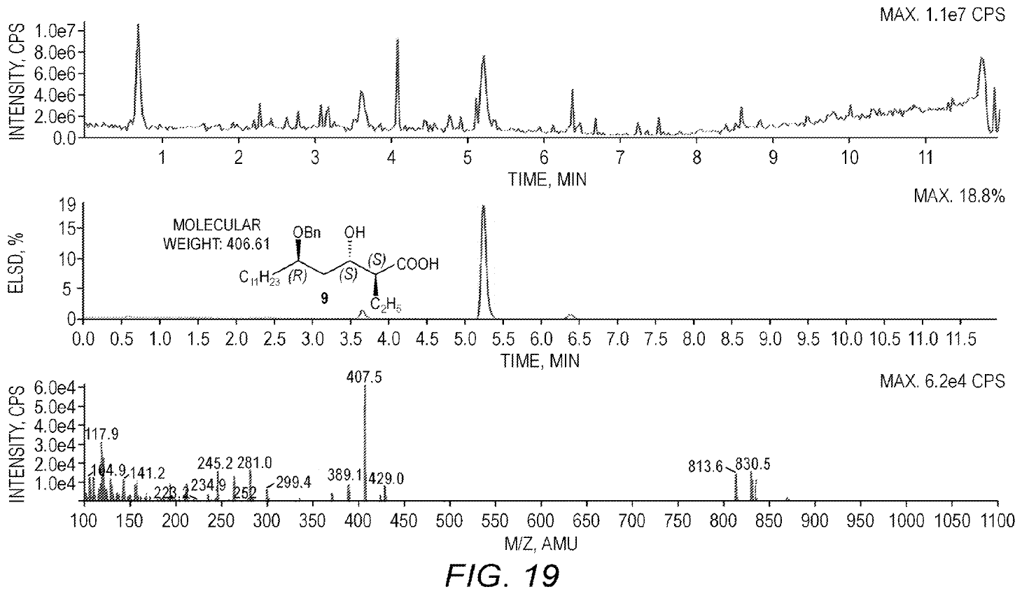

LCMS confirmed the reaction of INT 8-PEA to INT 9 was complete (See FIG. 19). The stirring was stopped to allow the layers to settle and the 2N HCl layer was separated. The organic layer was washed with saturated NaHCO₃ (3×1 L) to pH 8-9. The organic layer was then dried over anhydrous Na₂SO₄ (˜300 g) for 1 h, filtered and concentrated in vacuo to yield a thick brick red oil (430 g). The crude oil was redissolved in methyl acetate (1.5 L, Aldrich). The solution was washed with 0.5N HCl (1×500 mL, VWR), followed by water (2×500 mL) and brine (1×500 mL). The organic layer was dried over anhydrous Na₂SO₄ (˜100 g) for 1 h, then filtered and the Na₂SO₄ cake was washed with methyl acetate (500 mL). The combined filtrate (INT 9) was taken forward to the next step.

A 3-neck 5 L RBF was equipped with an overhead stirrer, cooling bath, argon inlet and outlet and a thermocouple/controller. INT 9 dissolved in methyl acetate (2 L) was transferred into a 5 L RBF. The reaction mixture was stirred and cooled to 5-10° C. (S)-(−)-alpha methylbenzylamine (95.4 g, Chem-Impex) was charged to the addition funnel and added to the reaction mixture dropwise while maintaining the temperature at 5-10° C. The resultant thick slurry was stirred at RT for 16 h.

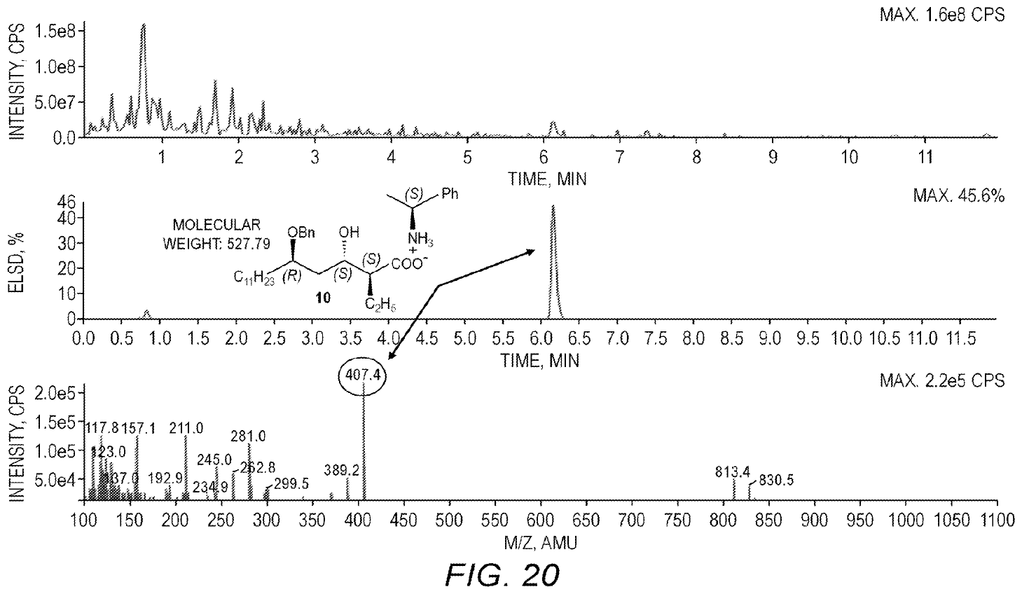

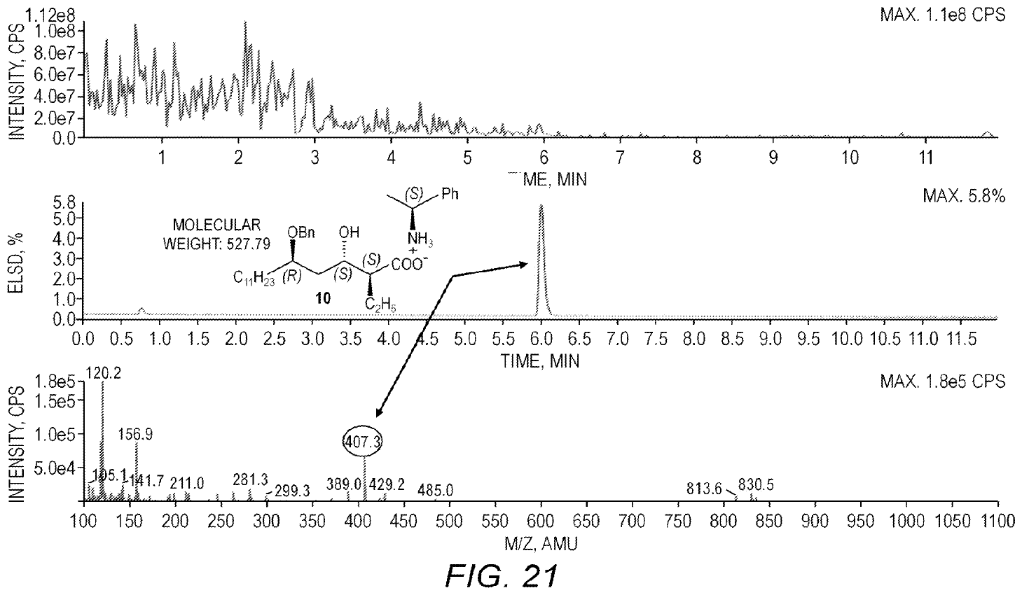

The slurry of crystals was cooled to 5-10° C. and stirred for 1 h. The crystals were filtered through coarse frit funnel and the solids were washed with cold methyl acetate (200 mL). The crystals were air dried for 1 h and then vacuum oven dried at RT until constant weight. INT 10 was isolated as a light yellow solid (196.4 g). The LS-MS spectrum is shown in FIG. 20.

### 3. Re-crystallization of INT 10

A 3-neck 5 L RBF was equipped with an overhead stirrer, heating mantle, argon inlet and outlet, and a thermocouple/controller. INT 10 (674 g) was transferred into 5 L RBF. Methyl acetate (2.7 L, 5V) was added to the pot and stirred at RT. The slurry mixture was heated to 50° C. to completely dissolve all the solid. Once the mixture became homogeneous, the heat was turned off and the mixture stirred overnight to cool to RT.

The crystals were cooled to 5-10° C. and stirred for 1 h. The crystals were filtered through a coarse frit funnel and the solids were washed with cold methyl acetate (500 mL). The crystals were air dried for 1 h and then vacuum oven dried at RT until constant weight. INT 10 was isolated as off white solid (589 g, 87% recovery). The LC-MS of recrystallized INT 10 is shown in FIG. 21.

### Example 12: Preparation Purified INT 9

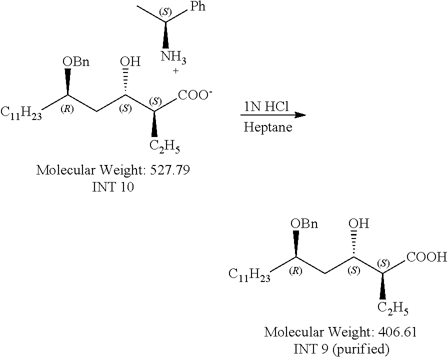

A 3-neck 12 L RBF was equipped with an overhead stirrer, cooling bath and a thermocouple/controller. INT 10 (580 g, 1.1 mol) was added to the flask followed by heptane (5.8 L, 10V) and stirred at RT. 1N HCl was prepared by using 2N HCl. 1N HCl (1160 mL, 2V) was added to the reaction mixture and stirred at RT for 16 h.

### 1. Work-up & Isolation

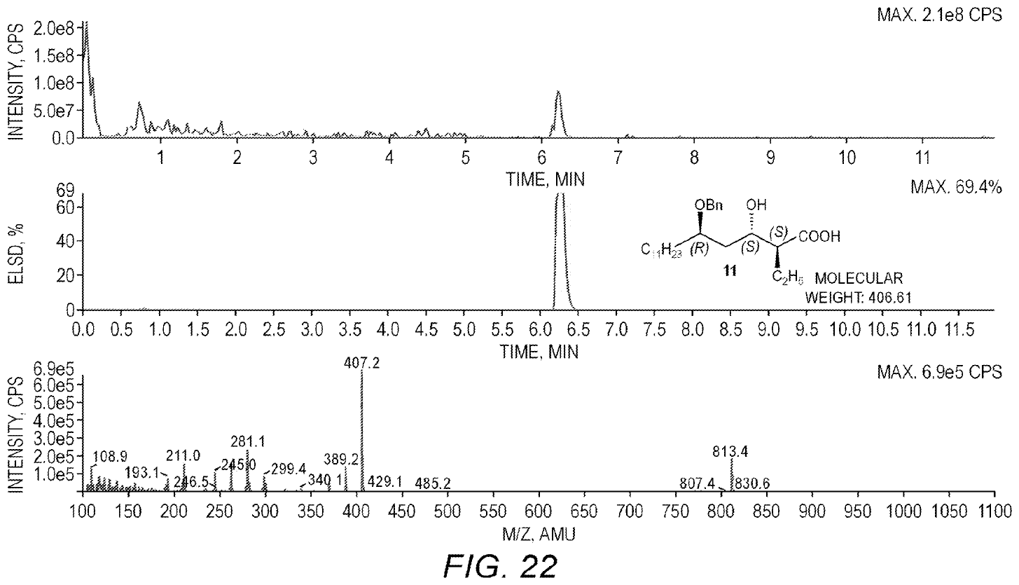

LCMS confirmed the reaction was complete (See FIG. 22). The stirring was stopped and the TN HCl layer was separated. The organic layer was washed with water (3×1 L) and dried over anhydrous Na₂SO₄ (˜300 g) for 1 h. The organic layer was filtered and concentrated in vacuo to yield an oil (460.7 g, >100%, contains traces of solvent).

### Example 13: Preparation of INT 11

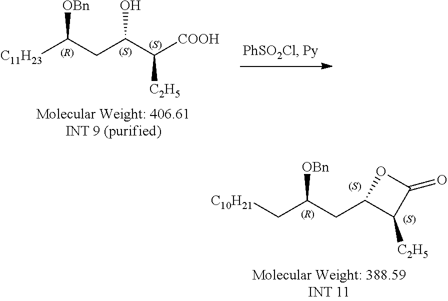

A 3-neck 12 L RBF was equipped with an overhead stirrer, cooling bath, argon gas inlet and outlet, and a thermocouple/controller. INT 9 (purified) (450 g, 1.11 mol) in a 22 L rotavap flask was dissolved with pyridine (4.5 L, 10V) and transferred to the 3-neck flask. The reaction mixture was cooled to <5° C. under argon. Benzenesulfonyl chloride (342 g, 1.75 eq, 1.94 mol, Aldrich) was charged to the 500 mL addition funnel. The reagent was added dropwise to the flask while maintaining the temperature below 5° C. The reaction was then stirred overnight at room temperature for 16 h.

### 1. Work-up & Isolation

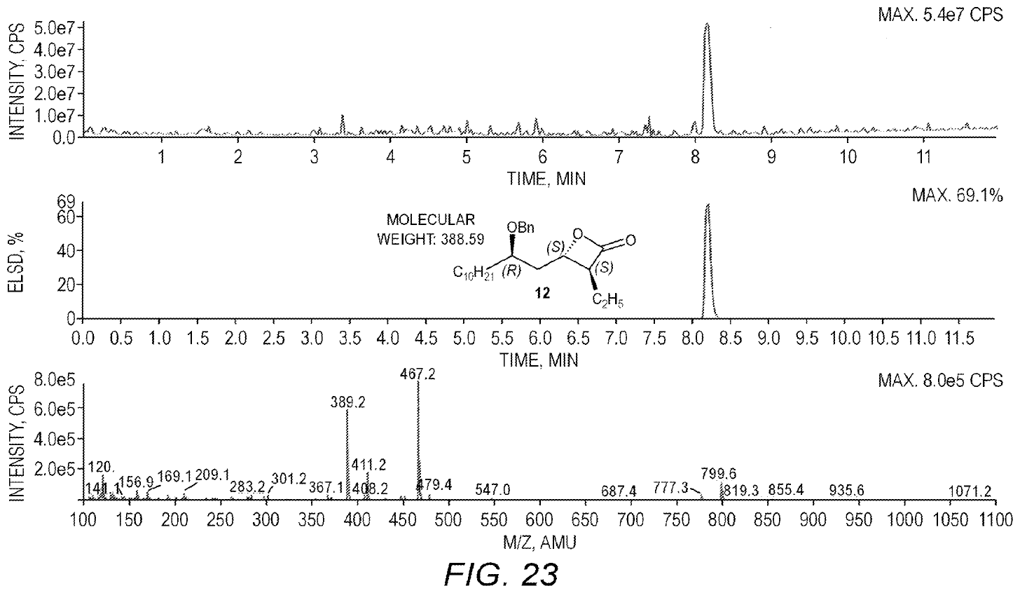

LCMS confirmed the reaction was complete (See FIG. 23). The reaction mixture was cooled to 5-10° C. Water (4.5 L) was added to the reaction while maintaining the temperature below 20° C. The reaction mixture was stirred for 30 minutes. The product was extracted into heptane (3×2 L). The combined organic layer was washed with 1N HCl (2×1.5 L) followed by 5% NaHCO₃ (2×1.5 L) and 10% NaCl (2×1.5 L). The organic layer was dried over anhydrous Na₂SO₄ (˜400 g, Aldrich) for 1 h. The organic layer was filtered and concentrated in vacuo to yield a brick red thick oil (423.8 g, 99%).

### Example 14: Preparation of INT 12

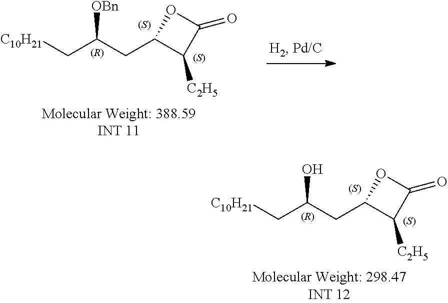

Using a set-up as shown in FIG. 10, a 3-neck 12 L RBF was equipped with an overhead stirrer, cooling bath, argon gas inlet and outlet and a thermocouple/controller. INT 11 (420 g, 1.08 mol) was added to the flask followed by THF (4.2 L, 10 vol.). The slurry was stirred under argon until a homogeneous solution was obtained. Under an argon atmosphere, 10% Pd/C (42 g, 10 wt %, Aldrich) was added to the flask. The flask was evacuated and refilled with hydrogen 3 times. Hydrogen was bubbled directly through the reaction mixture via gas diffusion sparger. The reaction was then stirred overnight at room temperature.

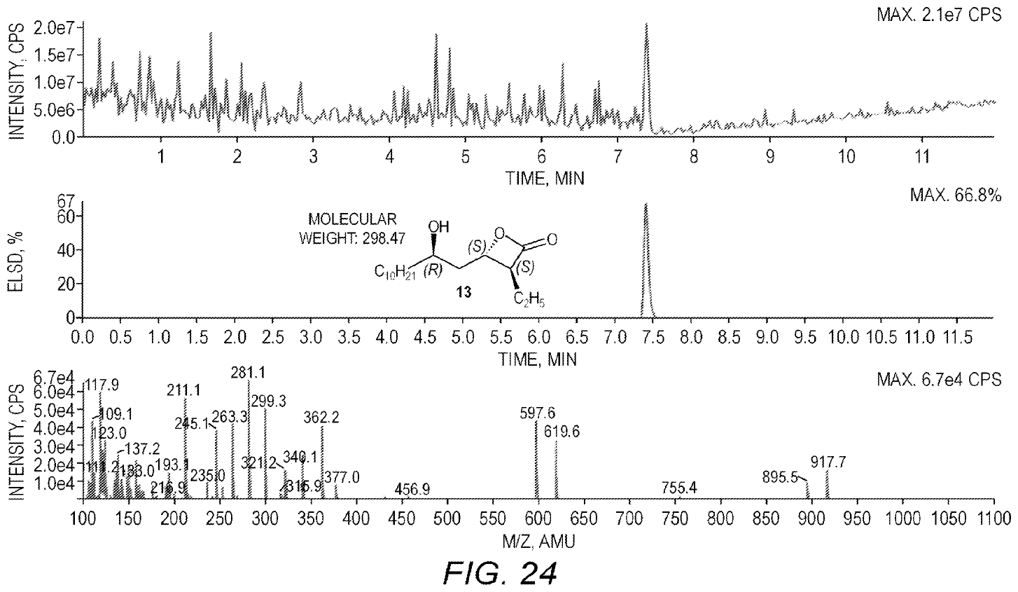

After 18 h stirring the hydrogen flow was stopped and argon was passed through the system for 5-10 min before the reaction was opened to atmosphere. A sample was then removed for IPC (LCMS). LCMS data shows completion of reaction and formation of INT 12 (See FIG. 24).

### 1. Celite Pad Preparation

A slurry of 100 wt % Celite (Sigma Aldrich) in minimal THF was prepared and added to a 2 L coarse frit. A piece of filter paper was placed on top of the slurry. The Celite was allowed to settle and light vacuum was applied to form a compact celite pad.

### 2. Pd/C Removal & Product Isolation

The reaction mixture was filtered through celite bed carefully without drying the Pd/C. The bed was washed with THF (3 L) and the combined filtrate was concentrated in vacuo using the 22 L rotavapor, yielding the crude product as an oil. After standing at room temperature the oil turned into off white solid (326.6 g, >100% yield).

### Example 15: Preparation of the Compound I-A

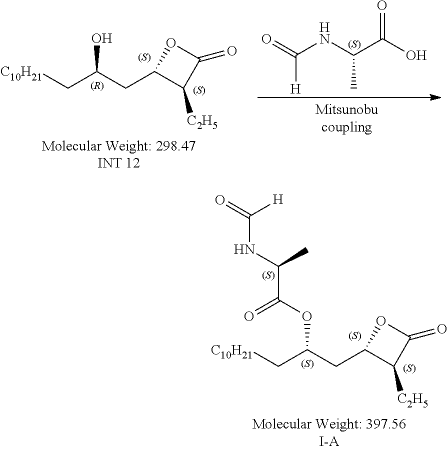

### 1. Alternative Reagent

Previous batches of Compound I-A utilized diisopropyl azodicarboxylate (DIAD) as a coupling reagent along with triphenylphosphine in the final step. The crude reaction mixtures for these steps were observed to contain diisopropyl hydrazinodicarboxylate (DIAD-H₂) as a major side product. In the case of the engineering batch subsequent chromatographic purification showed that almost 40% of the desired product coeluted with DIAD-H₂. To avoid such mixtures in future column purifications, di-tert-butyl azodicarboxylate (DBAD) was selected as an alternative reagent for the final step. The original rationale for switching to DBAD was the anticipation that the side product (DBAD-H₂) could be easily removed by degradation. The degradation process is shown in the following scheme.

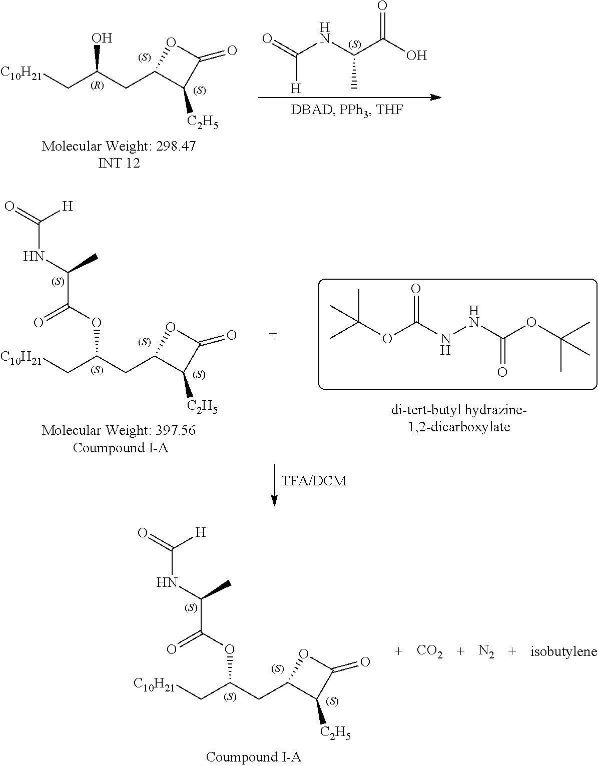

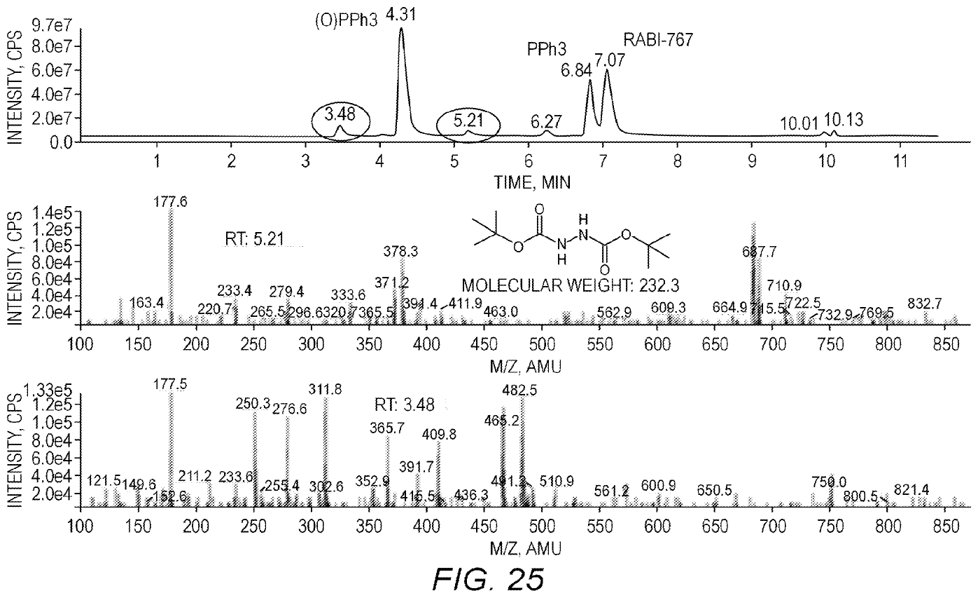

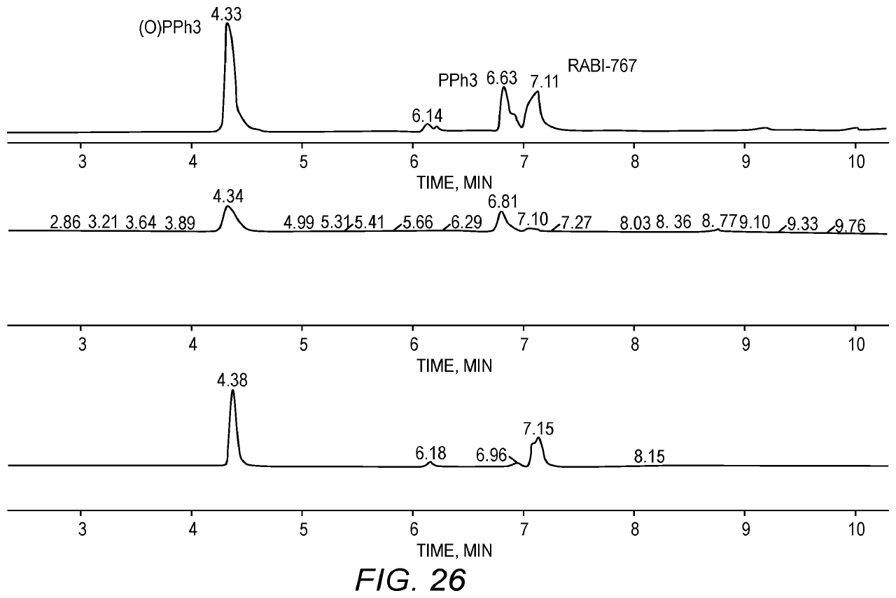

The expectation was that treatment of DBAD-H₂ in the reaction mixture with TFA/DCM at elevated temperatures should degrade the side product to CO₂ (gas), N₂ (gas), and isobutylene (gas) which could escape from the reaction mixture. INT 12 (300 mg) was used to test the DBAD activity for the Mitsunobu reaction and LCMS confirmed that the reaction worked using the modified conditions (FIG. 25). From 1 g of crude reaction mixture, 50 mg was treated with TFA/DCM (1:2) and heated at 55° C. for 1 h. LCMS (FIG. 26) showed that the DBAD-H₂ peak (RT:5.21) had disappeared without affecting the stability of Compound I-A.

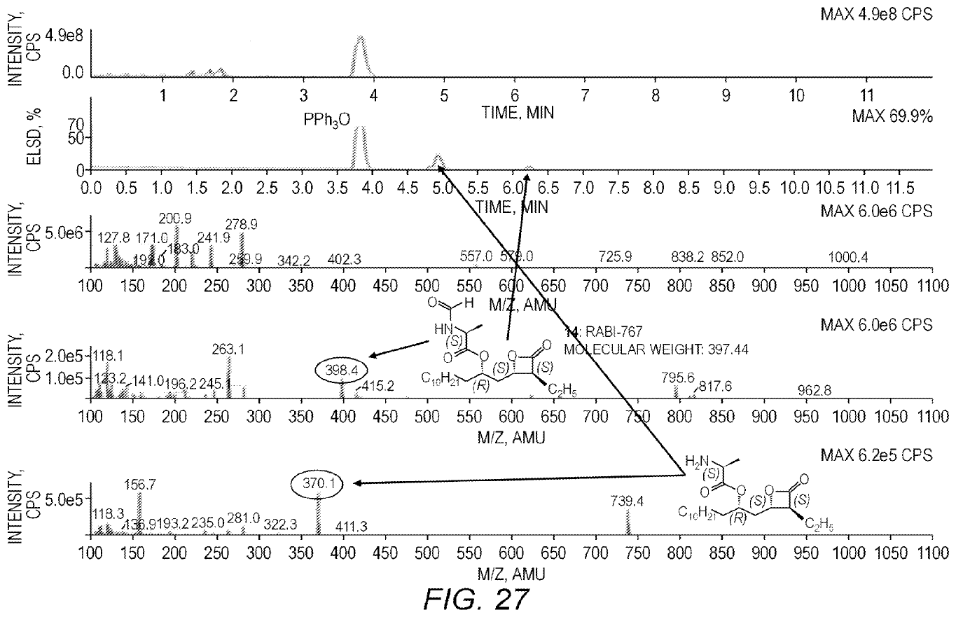

Another 30 g scale reaction was performed using the same procedure. On larger scale, LCMS showed that Compound I-A was partially decomposed during the attempted DBAD deprotection and the new impurity was identified as the deformylated analogue of Compound I-A (See FIG. 27). Under acidic conditions and at elevated temperatures, Compound I-A was decomposing along with DBAD-H₂. Hence this method was not deemed viable on larger scale.

However, another advantage of using the DBAD reagent in the Mitsunobu step was that the retention times of Compound I-A & DBAD-H₂ were well separated on TLC compared to Compound I-A & DIAD-H₂. It was therefore decided to use the DBAD reagent during the large scale Mitsunobu reaction.

### 2. Toxicology Batch

A 3-neck 12 L RBF was equipped with an overhead stirrer, cooling bath, 500 mL addition funnel, argon gas in and outlets and a thermocouple/controller. INT 12 (274 g, 0.918 mol, 1 eq), N-formyl L-alanine (139.7 g, 1.193 mol, 1.3 eq) and PPh₃ (288.9 g, 1.102 mol, 1.2 eq) were added to the flask. THF (2000 mL) was charged to the RBF and stirred at RT. The reaction mixture was partially dissolved and cooled to 5-10° C. and argon flow was initiated. Di-tert-butyl azodicarboxylate (DBAD, 253.7 g, 1.102 mol, 1.2 eq) was diluted with THF (700 mL) and charged to the 500 mL addition funnel. The solution was added to the reaction mixture dropwise by maintaining the temperature at 5-10° C. The addition funnel was rinsed with THF (40 mL) and the rinse added to the reaction mixture. The reaction mixture became a clear solution after the complete addition of DBAD. The reaction mixture was stirred at RT for 12 h under argon flow.

### 3. Work-Up & Initial Purification

TLC & LCMS confirmed the reaction was complete. The reaction mixture was transferred to a 10 L rotavapor flask and evaporated in vacuo to yield thick oil (971 g). Heptane (1000 mL) was added, and the resultant mixture was stirred at 10-15° C. for 2 h. A semi-solid was formed, which was allowed to settle after suspension of stirring. The heptane was decanted and the semi-solid was triturated with heptane (1000 mL). TLC showed the heptane layer contained less polar impurities and triphenyl phosphine oxide. MTBE (1000 mL) was added to the semi-solid and stirred at RT. The semi-solid turned into a free flowing solid and the solid was collected on a medium frit funnel. The solid was washed with MTBE (2×500 mL). TLC & LCMS confirmed that the solid was triphenyl phosphine oxide. The combined MTBE layer was evaporated in vacuo to yield 894 g crude product as an oil.

### 4. Column Purification

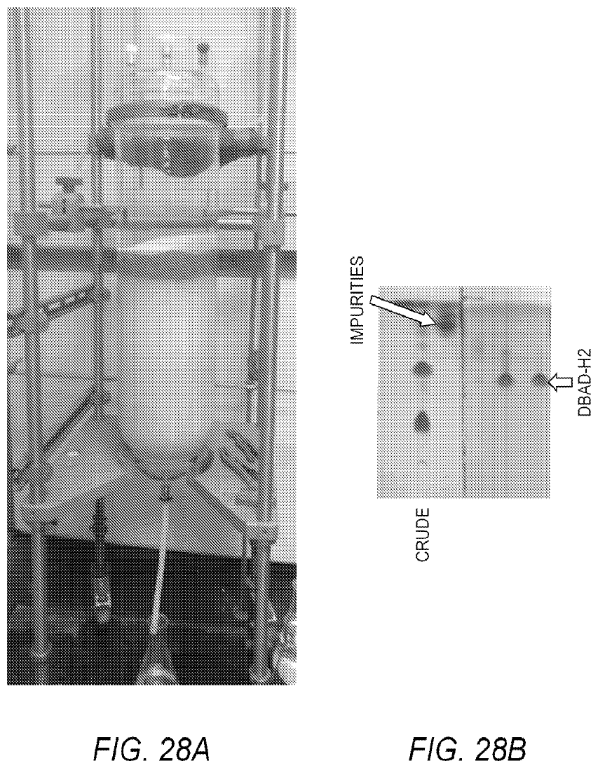

A large glass column (See FIG. 28A) was packed using silica gel (4.4 Kg, 5V. 60 Å, 230-400 mesh, Aldrich) and heptane. 893 g of crude Compound I-A was dissolved in MTBE (1000 mL) and added to 900 g of silica. The mixture was evaporated to yield the crude compound adsorbed on silica. The dry silica was loaded on top of a packed column, on top 1 cm sand and a filter paper was added. 1000 mL fraction volumes were collected.

Compound was eluted in the following manner. Ethyl acetate, heptane.
100% heptane—14 L
10% EA: heptane—25 L—upper impurities
15% EA: heptane—36 L—upper impurities+DBAD-H₂
20% EA: heptane—120 L—product
30% EA: heptane—120 L—product+lower impurities

During the 20% EA: heptane elution, the initial fractions contained a less polar impurity (minor) with product and the later fractions contained pure product. Only pure fractions by TLC (see FIG. 28B—mobile phase: 50:50 heptane/ethyl acetate, PMA stain) were collected and evaporated under vacuum.

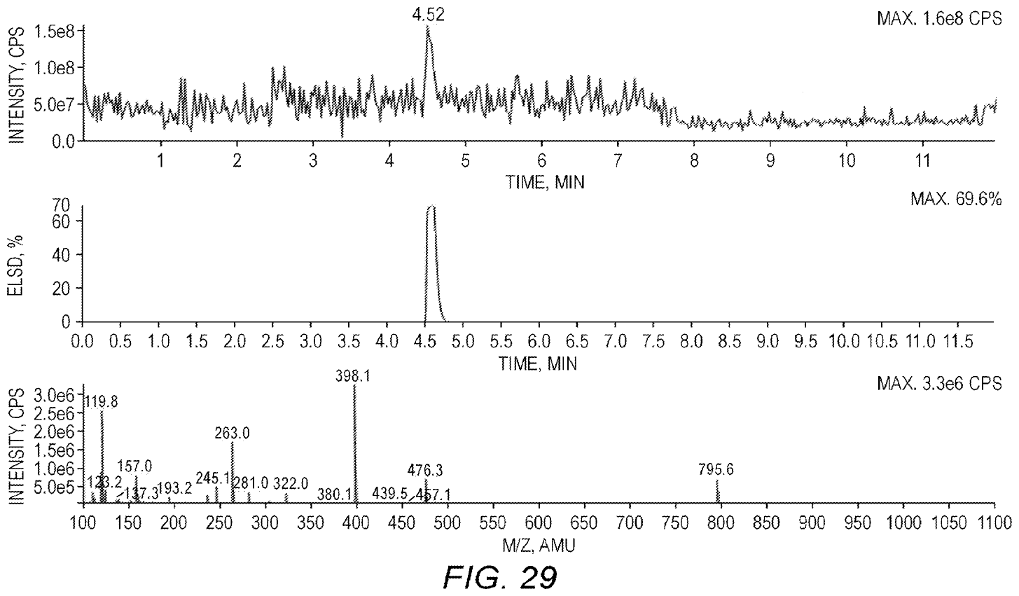

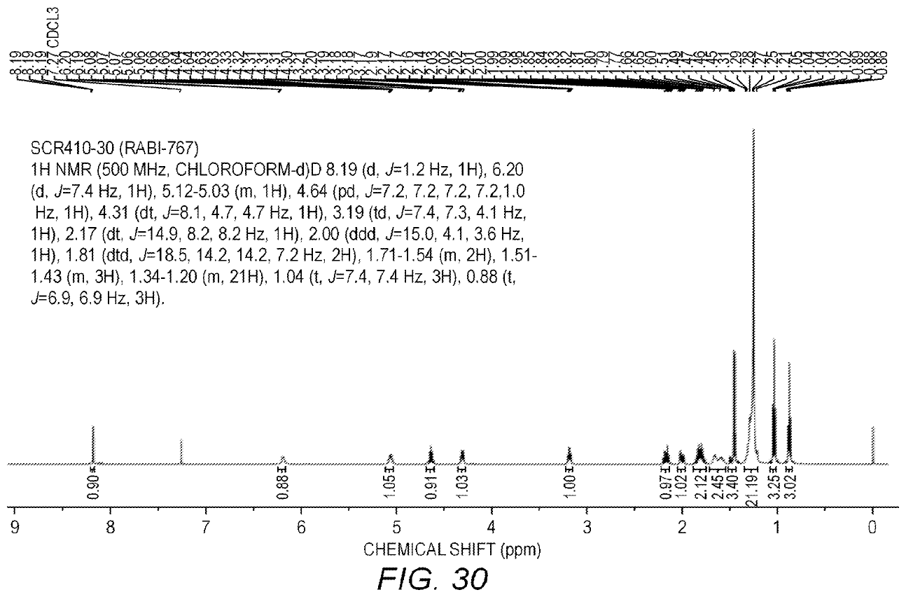

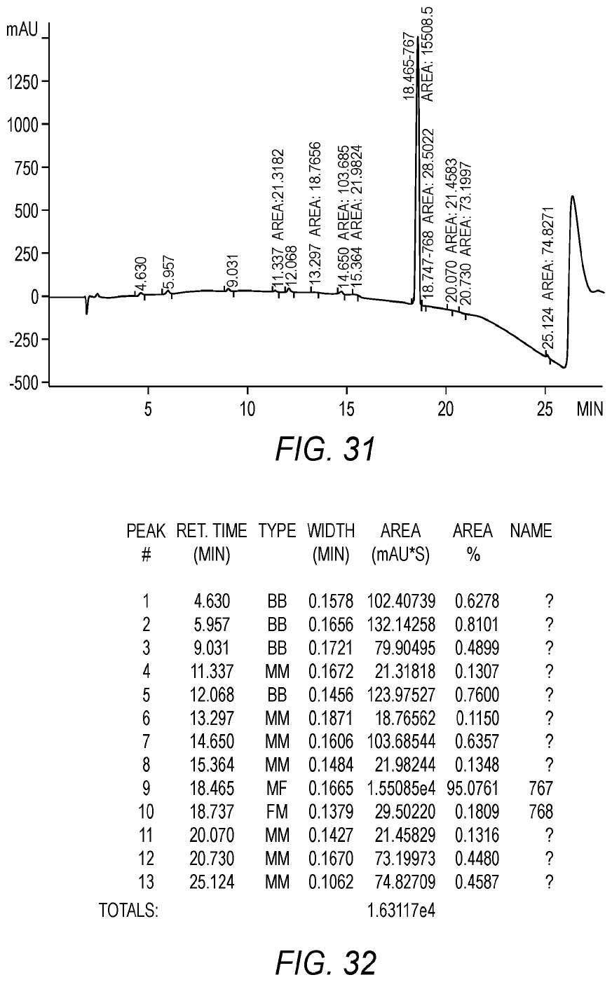

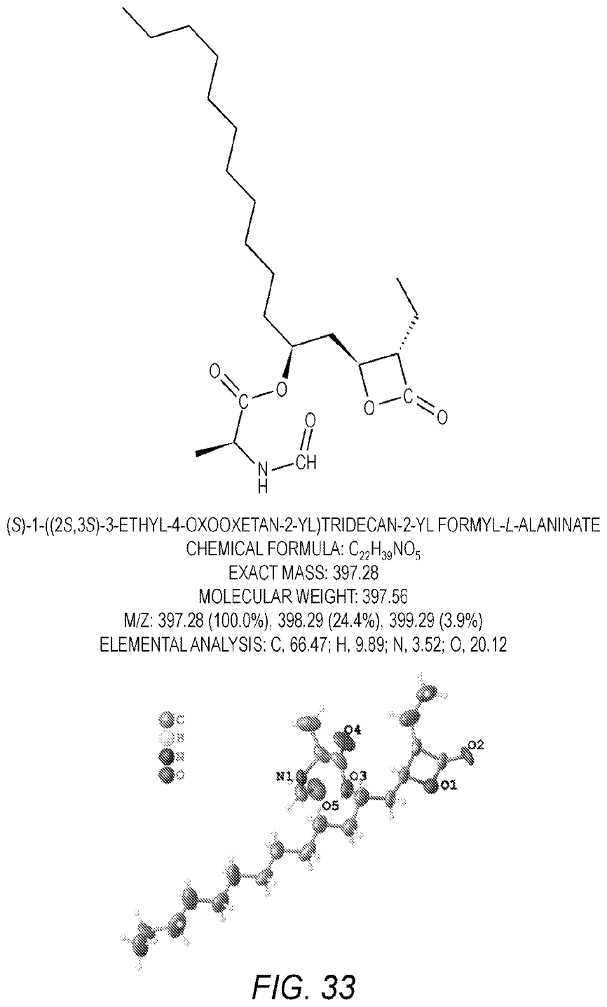

The combined pure fractions were evaporated in vacuo to yield an oil, which was seeded with Compound I-A (1.3 g) and kept in the freezer to solidify. The oil turned into a waxy off white solid (254 g, 70%) over the weekend (Compound I-A). Subsequent analysis by UPLC showed this material to be ca. 95% pure (AN at 205 nm). The LC-MS spectrum is shown in FIG. 29, the ¹H NMR spectrum is shown in FIG. 30, and HPLC is shown in FIGS. 31 and 32. The crystal structure of Compound I-A is shown in FIG. 33.

The foregoing is illustrative of the present invention and is not to be construed as limiting thereof. The invention is defined by the following claims, with equivalents of the claims to be included therein.

1. A method for synthesizing a compound of Formula I, 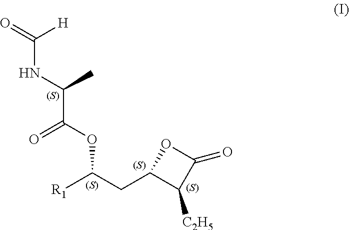
wherein R₁ is a C₅-C₁₅ alkyl (e.g., C₅-C₁₅ straight chain alkyl), the method comprising:
contacting a compound of INT 12 with N-formyl L-alanine, in the presence of di-tert-butyl azodicarboxylate (DBAD) and triphenylphosphine, to form the compound of Formula I:

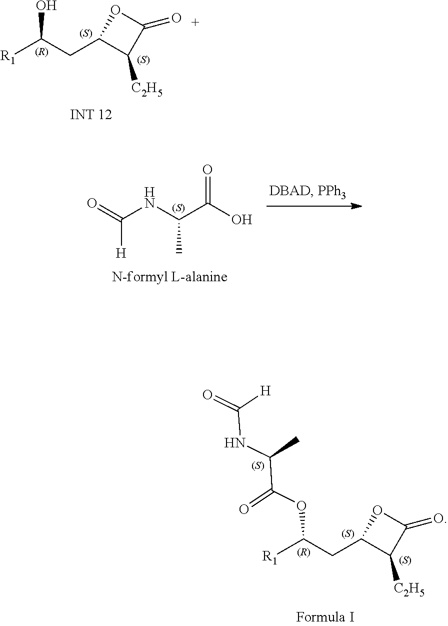

2. The method of claim 1, wherein R₁ is C₁₁ alkyl (e.g., C₁₁H₂₃).

3. The method of claim 1, wherein the contacting occurs in a tetrahydrofuran (THF) solvent.

4. The method of claim 1, wherein the contacting comprises the steps of:
(a) combining INT 12, N-formyl L-alanine, triphenylphosphine, and a solvent (e.g., THF) to form a reaction mixture;
(b) cooling the reaction mixture to a temperature in a specified temperature range below room temperature (e.g., 5-10° C.);
(c) dissolving DBAD in a solvent (e.g., THF);
(d) adding DBAD to the reaction mixture at a rate sufficient to maintain the temperature within the specified temperature range until the compound of Formula I is formed.

5. A method for synthesizing a compound of INT 2, comprising
contacting a compound of INT1 with a ruthenium (R)-BINAP catalyst and hydrogen gas in a reaction mixture in a pressure vessel to form a compound of INT 2:

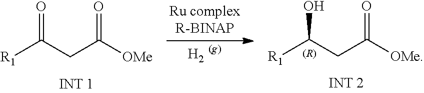

6. The method of claim 5, wherein the pressure of the hydrogen gas in the pressure vessel is less than or equal to 200 psi (e.g., in a range of 100 to 200 psi).

7. The method of claim 5, wherein a volume ratio of dead space to the reaction mixture in the pressure vessel is 3:1 or more.

8. The method of claim 5, wherein contacting comprises
(a) combining a ruthenium precatalyst (e.g., [RuCl₂benzene]₂) and R-BINAP to form the ruthenium (R)-BINAP catalyst;
(b) combining the INT 1 with a solvent (e.g., methanol) to form the reaction mixture;
(c) adding the ruthenium (R)-BINAP catalyst to the reaction mixture; and
(d) heating the reaction mixture (e.g., to 100° C.), to form INT 2.

9. A method for synthesizing a compound of Formula I, 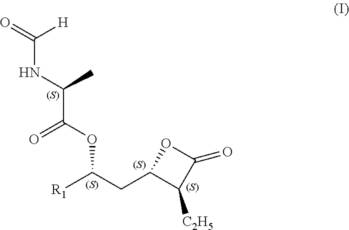
wherein R₁ is a C₅-C₁₅ alkyl (e.g., C₅-C₁₅ straight chain alkyl), the method comprising:
(a) contacting a compound of 1-A with a compound of 1-B, optionally in the presence of an alkali metal (e.g., magnesium), to form a compound of INT 1: 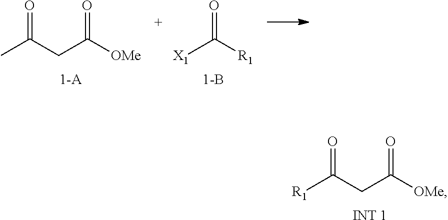
wherein X₁ is a halo (e.g., Cl or Br);
(b) contacting the compound of INT1 with a ruthenium (R)-BINAP catalyst and hydrogen gas to form the compound of INT 2: 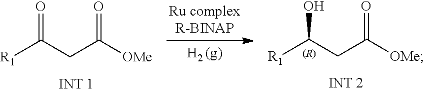
(c) contacting the compound of INT 2 with a compound of 3-A to form a compound of INT 3: 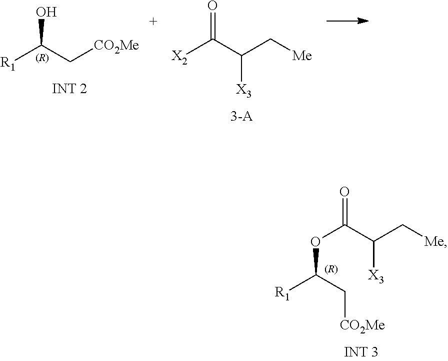
wherein X₂ and X₃ are each independently halo (e.g., Br);
(d) contacting the compound of INT 3 with a Grignard reagent to form a compound of INT 4: 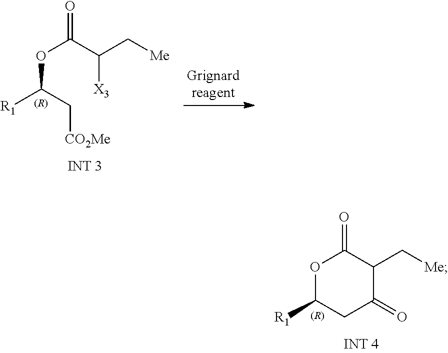
(e) contacting the compound of INT 4 with hydrogen gas in the presence of a Raney Ni catalyst to form a compound of INT 5: 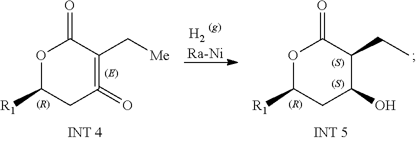
(f) contacting the compound of INT 5 with a first protecting agent to form a compound of INT 6: 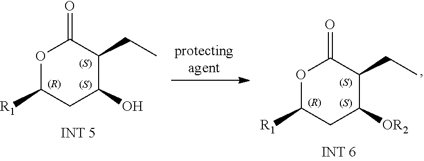
wherein R₂ is a protecting group (e.g., THP);
(g) contacting the compound of INT 6 with a hydroxide (e.g., from a hydroxide salt such as NaOH) to form a compound of INT 7: 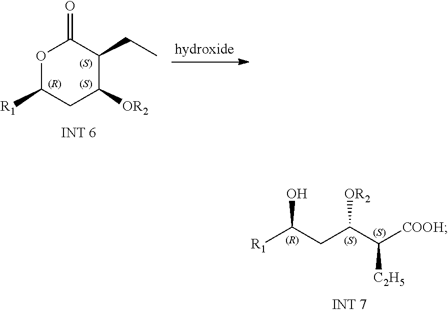
(h) protecting a free hydroxy group on a compound of INT 7 by reacting INT 7 with a second protecting agent to form a compound of INT 8, and then deprotecting a protected hydroxy group on the compound of INT 8 by contacting INT 8 with acid to form the compound of INT 9: 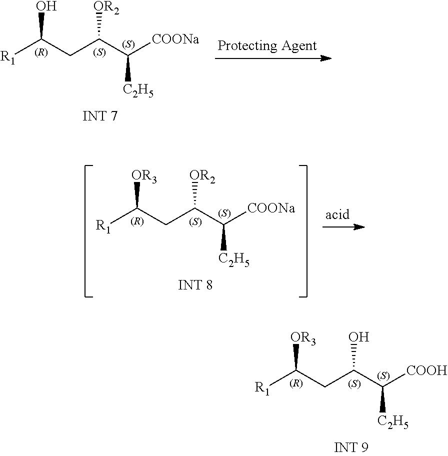
wherein R₃ is a protecting group (e.g., benzyl);
(i) optionally, purifying the compound of INT 9 by contacting the compound of INT 9 with (S)-(−)-1-phenyethylamine to form a compound of INT 10: 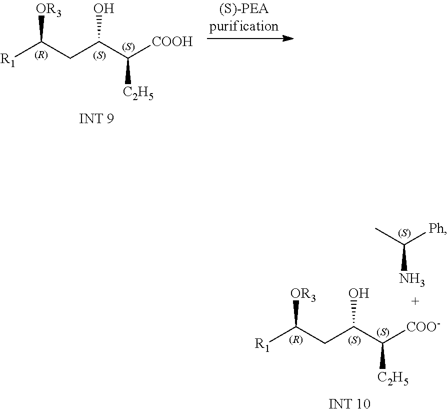
crystallizing and isolating INT 10A, and then contacting INT 10 with an acid (e.g., HCl) to form INT 9 (purified): 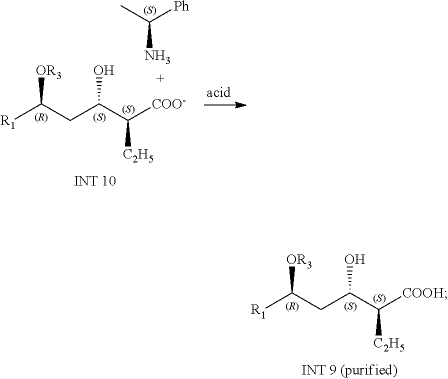
(j) dehydrating the compound of INT 9 or the compound of INT 9 (purified) with a dehydrating agent to form a compound of INT 11: 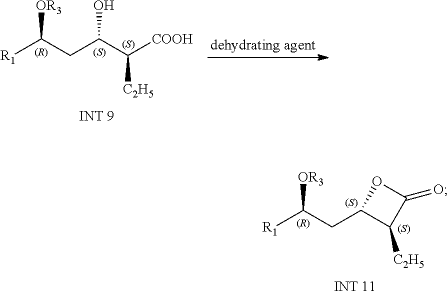
(k) deprotecting the compound of INT 11 to form a compound of INT 12: 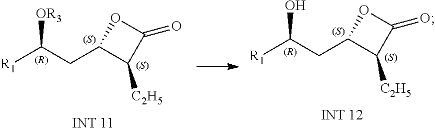
and
(l) contacting the compound of INT 12 with N-formyl L-alanine, in the presence of di-tert-butyl azodicarboxylate (DBAD) and triphenylphosphine, to form a compound of Formula I:

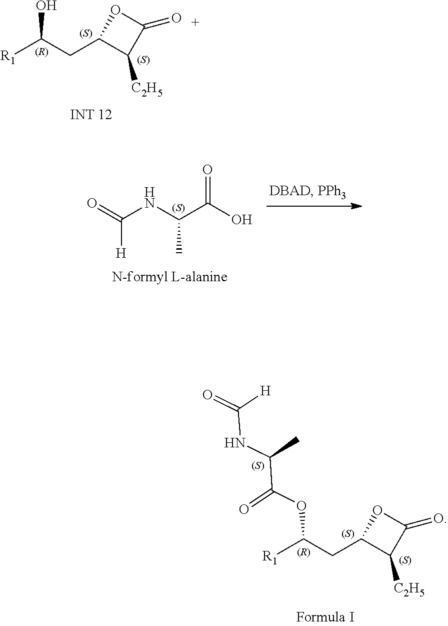

10. The method of claim 9, wherein in step (b), contacting the compound of INT 1 with the ruthenium (R)-BINAP catalyst and hydrogen gas occurs in a reaction mixture in a pressure vessel, and wherein a ratio of the volume of dead space to the volume of the reaction mixture in the pressure vessel is 3:1 or more.

11. The method of claim 9, wherein in step (b), the pressure of the hydrogen gas is less than 200 psi (e.g., in a range of 100 to 200 psi).

12. The method of claim 9, wherein in step (c), the contacting occurs in the presence of a dimethylaminopyridine (DMAP) and aqueous potassium bicarbonate (KHCO₃) in a two-phase reaction.

13. The method of claim 9, wherein in step (d), the Grignard reagent comprises tert-butylMgBr.

14. The method of claim 9, wherein in step (e) the Raney Ni catalyst is freshly prepared, and the pressure of the hydrogen gas is in a range of 0.5 to 2 atm.

15. The method of claim 9, wherein in step (f), the protecting agent is 3,4-dihydro-2H-pyran (DHP) and R₂ is a tetrahydropyran (THP) group.

16. The method of claim 9, wherein in step (i), after crystallization and isolation of the compound of INT 10, the compound of INT 10 is contacted with an acid to form purified INT 9.

17. The method of claim 9, wherein the dehydrating agent in step (j) comprises benzene sulfonyl chloride.

18. The method of claim 9, wherein in step (k), the deprotecting comprises a debenzylation reaction, optionally using Pd/C and hydrogen gas.

19.-22. (canceled)
